(19) United States
(12) Patent Application Publication
(10) Pub. No.: US 2024/0301355 A1
(43) Pub. Date: Sep. 12, 2024

(54) METHODS OF INDUCING CARDIAC CELL PROLIFERATION AND INDUCING HEART REGENERATION

(71) Applicant: Wisconsin Alumni Research Foundation, Madison, WI (US)

(72) Inventors: Ahmed Mahmoud, Middleton, WI (US); Timothy Kamp, Madison, WI (US); Youngsook Lee, Madison, WI (US)

(73) Assignee: Wisconsin Alumni Research Foundation, Madison, WI (US)

(21) Appl. No.: 18/596,320

(22) Filed: Mar. 5, 2024

Related U.S. Application Data
(60) Provisional application No. 63/488,657, filed on Mar. 6, 2023.

(51) Int. Cl. C12N 5/077; A61K 35/34; A61P 9/10; C12N 15/86

(52) U.S. Cl. CPC C12N 5/0657; A61K 35/34; A61P 9/10; C12N 15/86; C12N 2501/998; C12N 2510/00; C12N 2750/14143

(57) ABSTRACT

Methods of inducing mammalian cardiac cell proliferation by ectopically introducing an LRRC10 protein in a mammalian cardiac cell to thereby induce proliferation of the cardiac cell. The methods can be employed to induce heart regeneration, for example, after cardiac injury.

### CROSS-REFERENCE TO RELATED APPLICATIONS

Priority is hereby claimed to U.S. Provisional Application 63/488,657, filed Mar. 6, 2023, which is incorporated herein by reference in its entirety.

### STATEMENT REGARDING FEDERALLY SPONSORED RESEARCH

This invention was made with government support under HL134764 and HL155617 awarded by the National Institutes of Health and under W81XWH-22-1-0094 awarded by the ARMY/MRDC and under EEC1648035 awarded by the National Science Foundation. The government has certain rights in the invention.

### SEQUENCE LISTING

The instant application contains a Sequence Listing which has been submitted in XML format and is hereby incorporated by reference in its entirety. The XML copy, created on Mar. 4, 2024, is named USPTO-09824493-P230238US02-SEQ_LIST and is 3,713 bytes in size.

### FIELD OF THE INVENTION

The invention is directed to a methods of inducing mammalian cardiac cell proliferation and inducing heart regeneration.

### BACKGROUND

Myocardial infarction and heart failure are some of the leading causes of mortality and hospitalization worldwide (Savarese et al. 2017, Bui et al. 2011). Almost 300,000 individuals each year experience recurrent heart attacks in the United States alone (Dargie et al. 2005, Go et al. 2014), and the prevalence of ischemic heart disease is projected to rise to about 40.5% of the USA population by 2030 (Go et al. 2014). Traditional approaches for dealing with end-stage heart failure are often not feasible, due to the limited number of hearts available for transplantation. Preclinical trials have reported improvements in patient outcomes (Hajjar et al. 2008, Greenburg et al. 2016), but prognosis remains poor. There is, therefore, an urgent need for new approaches to the prevention and treatment of heart failure.

During heart failure, billions of cardiomyocytes are progressively lost, and fibrotic non-functional scar tissue develops, significantly reducing the pumping capacity of the heart muscle. The remaining cardiomyocytes have a limited intrinsic regenerative capacity and cannot, therefore, replace the lost cardiomyocytes. Cardiac regeneration studies have shown that dividing cardiomyocytes are abundant in the fetus, but rapidly lost after birth (Porrello et al. 2011). Cell-based therapies with exogenous cells, such as bone marrow cells, cardiac progenitor cells, and other self-renewing stem cells, have been developed to improve heart function. However, little meaningful improvement has been reported for these treatments, owing to the limited interaction between the various progenitor cells and the myocardium environment during myocardial infarction.

In the last two decades, our understanding of the molecular pathways and genes involved in the disease has improved, and gene therapy has emerged as a possible treatment for heart failure. Given the limited site specificity of pharmacological inhibitors, gene therapy is an exciting prospect for more precise targeting of the signaling pathways involved in disease progression. The gene therapy approaches currently being developed for heart failure aim to increase the proliferation or contractility of endogenous cardiomyocytes; to reprogram cardiac fibroblasts to develop into beneficial cardiac cell types, such as endothelial cells or cardiomyocytes; and to increase capillary density by activating endogenous endothelial cells or progenitors. Recent studies have reported reactivation of the cardiomyocyte cell cycle following protein delivery to the myocardium, either by direct injection or via patch delivering the protein to the epicardium. Cardiomyocyte proliferation has been reported following the delivery of NRG1 protein via intraperitoneal (i.p.) injections (Engel et al. 2005), intramyocardial injections of agrin (Bassat et al. 2017), or the delivery of follistatin-like 1 to the epicardium (Wei et al. 2015). Furthermore, the proliferation of adult cardiomyocytes has been observed following transfection with an adenovirus encoding a dominant-negative p38 mitogen-activated protein kinase (MAPK) (Engel et al. 2006) or an extracellular matrix component, periostin (Kuhn et al. 2007). The adeno-associated virus (AAV)-mediated delivery of Serca2a (del Monte et al. 2007) or SUMO (Kho et al. 2011) has been shown to improve cardiac function post-MI and in heart failure condition via elevation of endogenous cardiomyocytes contractility.

Additional gene therapy approaches for addressing other aspects of heart disease, heart failure, and cardiac injury generally are needed.

### SUMMARY OF THE INVENTION

One aspect of the invention is directed to methods of inducing mammalian cardiac cell proliferation. The methods preferably comprise ectopically introducing an LRRC10 protein in a mammalian cardiac cell to thereby induce proliferation of the cardiac cell.

Another aspect of the invention is directed to an LRRC10 protein or an ectopic agent that induces expression of an LRRC10 protein, for use in ectopically introducing the LRRC10 protein in a mammalian cardiac cell to thereby induce proliferation of the cardiac cell. In some versions, the ectopic agent is a recombinant nucleic acid encoding an LRRC10 protein.

Another aspect of the invention is directed to a use of an LRRC10 protein or an ectopic agent that induces expression of an LRRC10 protein for ectopically introducing the LRRC10 protein in a mammalian cardiac cell to thereby induce proliferation of the cardiac cell. In some versions, the ectopic agent is a recombinant nucleic acid encoding an LRRC10 protein.

Another aspect of the invention is directed to an LRRC10 protein or a recombinant nucleic acid encoding an LRRC10 protein, for use in ectopically introducing the LRRC10 protein in a mammalian cardiac cell to thereby induce proliferation of the cardiac cell.

Another aspect of the invention is directed to a use of an LRRC10 protein or a recombinant nucleic acid encoding an LRRC10 protein for ectopically introducing the LRRC10 protein in a mammalian cardiac cell to thereby induce proliferation of the cardiac cell.

In some versions, the cardiac cell comprises a cardiomyocyte.

In some versions, the LRRC10 protein is at least 80%, at least 85%, at least 90%, or at least 95% identical to SEQ ID NO:1.

In some versions, the ectopically introducing comprises ectopically expressing the LRRC10 protein in the cell. In some versions, the ectopic expression comprises expressing the LRRC10 protein from a recombinant nucleic acid encoding the LRRC10 protein within the cell. Some versions the ectopic expression comprises delivering an exogenous nucleic acid encoding the LRRC10 protein in the cardiac cell.

In some versions, the cardiac cell comprises a plurality of cardiac cells. In some versions, the cardiac cell is comprised within cardiac tissue.

In some versions, the cardiac cell is comprised within a heart. In some versions, the heart comprises a cardiac injury. In some versions, the cardiac injury comprises a myocardial lesion. In some versions, the myocardial lesion is selected from the group consisting of a myocardial infarct, decreased myocardial thickness, fibrosis, dilated cardiac chamber, increased left ventricular end-diastolic diameter, increased left ventricular end-systolic diameter, and any combination thereof. In some versions, the cardiac injury comprises a functional defect. In some versions, the functional defect is selected from the group consisting of reduced ejection fraction, reduced fractional shortening, reduced end-systolic elastance and any combination thereof. In some versions, the LRRC10 protein is introduced (e.g., expressed or delivered) in an amount and for a time effective to elicit an amelioration of the cardiac injury.

In some versions, the LRRC10 protein is introduced (e.g., expressed or delivered) in an amount and for a time effective to elicit an improvement in cardiac structure. In some versions, the improvement in cardiac structure is selected from the group consisting of a decrease in myocardial lesion size, an increase in myocardial thickness, decreased fibrosis, a decrease in left ventricular end-diastolic diameter, a decrease in left ventricular end-systolic diameter, an increase in coronary artery formation, an increase in capillary density, an increase in revascularization, and any combination thereof.

In some versions, the LRRC10 protein is introduced (e.g., delivered or expressed) in an amount and for a time effective to elicit an improvement in cardiac function. In some versions, the improvement in cardiac function is selected from the group consisting of an increase in an increase in ejection fraction, an increase in fractional shortening, an increase in end-systolic elastance, and any combination thereof.

In some versions, the cardiac cell is comprised within a subject. In some versions, the subject is suffering or has suffered from a cardiac event or a chronic heart condition. In some versions, the subject is suffering or has suffered from a cardiac event. In some versions, the cardiac event is selected from the group consisting of cardiac ischemia, cardiac ischemia-reperfusion, myocardial infarction, myocarditis, blunt trauma, and any combination thereof. In some versions, the subject is suffering or has suffered from a chronic heart condition. In some versions, the subject is suffering from or has suffered from heart failure. In some versions, the heart failure comprises heart failure with reduced ejection fraction. In some versions, the subject has a myocardial infarct.

Some versions comprise administering a nucleic-acid delivery reagent comprising an ectopic nucleic acid to the subject.

Another aspect of the invention is directed to methods of inducing cardiac regeneration in a mammalian subject suffering from cardiac injury. The methods preferably comprise ectopically introducing an LRRC10 protein comprising an amino acid sequence at least 80% identical to SEQ ID NO:1 in cardiac tissue cells in the subject to thereby induce cardiac regeneration in the subject.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1J. Cardiac regeneration is inhibited in the neonatal Lrrc10^{-/-} mouse with disruption of the transcriptional landscape following injury. Control and Lrrc10^{-/-} neonatal mice underwent a sham (SH) or myocardial infarction (MI) surgery at P1 and were analyzed at 7-, 14-, or 21 days post-surgery (DPS). (FIG. 1A) Mason's trichrome staining (viable tissue in red; scar tissue in blue) at 21DPS (n=5); scale bar=1 mm. (FIG. 1B) Analysis of Heart Weight to Body Weight (BW) ratio (n=12). (FIG. 1C) Analysis of cardiomyocyte mitosis with pH3 and cTnT at 7DPS (n=7); scale bar=10 uM. (FIG. 1D) Analysis of cytokinesis by symmetrical localization of Aurora B with cTnT at 7DPS (SH n=3; MI n=7); arrowhead indicates Aurora B localization between cardiomyocytes; scale bar=10 uM. (FIG. 1E) Percentage of mononucleated (Mono), binucleated (Bi), and multinucleated (Multi) cardiomyocytes per heart at 14DPS (WT n=4; KO n=5); scale bar=20 uM. (FIG. 1F) Quantification of cardiomyocyte size by Wheat Germ Agglutinin (WGA) at 21DPS (n=5); scale bar=25 uM. Hearts were collected from WT and Lrrc10^{-/-} mice at 7 days after sham or MI surgery, processed for bulk-tissue RNA-sequencing (n=4 per group), and the transcriptome of differentially expressed genes (DEGs) was identified. (FIG. 1G) Heatmap generated by k-means clustering of DEGs and divided into 8 clusters; colored scale is related to z-score. (FIG. 1H) Clusters 1, 3, 4, and 7 show a unique transcriptome in Lrrc10^{-/-} hearts. (FIGS. 1I and 1J) Gene Ontology (GO) analysis highlights LRRC10 dysregulated pathways related to muscle contraction (Cluster 1, FIG. 1I) and cell metabolism (Cluster 4, FIG. 1J). *P<0.05, **P<0.01, ****P<0.0001. Error bars presented as S.E.M.

FIG. 2. Lrrc10^{-/-} hearts show increased scar tissue post-MI. Masson's Trichrome staining was performed on control (WT) and Lrrc10^{-/-} heart sections at 21 days post (a) sham (SH) and (b) MI surgery (n=5); scale bar=1 mm.

FIGS. 3A-3F. Lrrc10^{-/-} hearts show no change in proliferation, asymmetrical cell division, or sham nucleation counts. At 7 days post-sham or post-MI surgery, there was no difference in cardiomyocyte (CM) proliferation between (FIG. 3A) WT and (FIG. 3B) Lrrc10^{-/-} groups, shown as representative sections (scale bar=500 μm) and (a′, b′) insets (scale bar=50 μm); arrowheads indicate pH3+ CMs. (FIG. 3C) WT and (FIG. 3D) Lrrc10^{-/-} hearts show no difference in Aurora B localization, shown as representative sections (scale bar=500 μm) and insets of (c′, d′) symmetrical (scale bar=20 μm) or (c′, d′) asymmetrical CM division (scale bar=20 μm); arrowhead indicates Aurora B localization between CMs. (FIG. 3E) Post-MI or post-sham surgery, Lrrc10^{-/-} hearts show no difference in asymmetrical CM division compared to WT hearts (scale bar=10 μm). (FIG. 3F) Nucleation counts of mono-, bi-, and multinucleated CMs show no difference between WT and Lrrc10^{-/-} mice at 14 days post-sham surgery.

FIGS. 4A and 4B. Transcriptomic analysis shows unique signature between control and Lrrc10^{-/-} hearts post-MI. Bulk RNA sequencing of control and Lrrc10^{-/-} mice that underwent a sham (SH) or MI surgery at PL. (FIG. 4A) Principal Component Analysis (PCA) shows separation between different groups. (FIG. 4B) Pearson correlation shows clustering of transcriptomic profiles.

FIGS. 5A-5G. LRRC10 overexpression rescues heart regeneration in Lrrc10^{-/-} mice. (FIG. 5A) Control and Lrrc10^{-/-} neonatal mice were treated with a single-dose of AAV9-cTnT-GFP control or AAV9-cTnT-LRRC10 rescue viral vector before undergoing sham or MI surgery at P1. (FIG. 5B) Efficient transfection shown by GFP expression in WT P8 mice treated with or without AAV9-cTnT-GFP at P0 (n=3). Hearts were analyzed for regeneration hallmarks at 7-, 14- and 28-days post-MI (DPMI). (FIG. 5C) Cytokinesis measured by symmetrical Aurora B within cardiomyocytes, marked by cTnT at 7DPMI (n=4). (FIG. 5D) Nucleation percentages of mononucleated (Mono), binucleated (Bi), and multinucleated (Multi) cardiomyocytes (WT and KO controls n=9; KO rescue n=8) at 14DPMI. (FIG. 5E) Masson's trichrome analysis at 28DPMI (n=4); scale bar=1 mm. (FIG. 5F) Heart weight to body weight (BW) ratios at 28DPMI (WT control n=9; KO control n=8; KO rescue n=6). (FIG. 5G) Echocardiography of ejection fraction (EF) and fractional shortening (FS) at 28DPMI (WT control n=9; KO control n=8; KO rescue n=6). *P<0.05, **P0.01, ****P<0.0001. Error bars presented as S.E.M.

FIG. 6. Viral control shows successful reporter expression in the postnatal heart. AAV9-cTnT-GFP control vector was injected into P0 mice and hearts were

collected at 7 days post-injections. Images were captured of the (a) whole-mount and (b) sectioned hearts (n=3). Scale bar=1 mm.

FIG. 7. Asymmetrical cytokinesis is unchanged between Lrrc10^{-/-} mice treated with AAV9-cTnT-GFP or AAV9-cTnT-LRRC10 rescue vector. AAV9-cTnT-GFP (control) or AAV9-cTnT-LRRC10 (rescue) vector was injected into P0 mice. MI surgery was performed at P1, and asymmetrical cytokinesis was measured by Aurora B at 7 days post-MI. No difference in asymmetric cardiomyocyte (CM) division was identified between Lrrc10^{-/-} hearts treated with control or rescue vector.

FIG. 8. Lrrc10^{-/-} mice treated with AAV9-cTnT-LRRC10 rescue vector promotes regeneration post-MI. Masson's Trichrome staining of heart sections at 21 days post-MI from WT and Lrrc10^{-/-} (KO) mice treated at P0 with (a-b) control AAV9-cTnT-GFP or (c) rescue AAV9-cTnT-LRRC10 vector (n=4); scale bar=1 mm.

### DETAILED DESCRIPTION OF THE INVENTION

One aspect of the invention is directed to methods of inducing cardiac cell proliferation. The methods preferably comprise ectopically introducing a leucine-rich repeat containing 10 (LRRC10) protein in the cardiac cell to thereby induce proliferation of the cardiac cell.

“Induce” as used herein in this context refers to increasing from a baseline level or rate.

“Proliferation” as used herein in this context refers to an increase in the number of viable cells as a result of cell growth and cell division.

“LRRC10 protein” as used herein refers to the wild-type human LRRC10 protein or homologs or variants thereof having an LRRC10 activity described herein, such as inducing cardiac cell proliferation.

In some versions, the LRRC10 protein comprises an amino acid sequence at least 80% identical to SEQ ID NO:1. SEQ ID NO:1 is the amino acid sequence of the wild-type human LRRC10 protein:

(SEQ ID NO: 1)
MGNTIRALVAFIPADRCQNYVVRDLREMPLDKMVDLSGSQLRRFP
LHVCSFRELVKLYLSDNHLNSLPPELGOLONLQILALDENNFKAL
PQVVCTLKOLCILYLGNNKLCDLPSELSLLONLRTLWIEANCLTO
LPDVVCELSLLKTLHAGSNALRLLPGQLRRLOELRTIWLSGNRLT
DFPTVLLHMPFLEVIDVDWNSIRYFPSLAHLSSLKLVIYDHNPCR
NAPKVAKGVRRVGRWAEETPEPDPRKARRYALVREESQELQAPVP
LLPPINS

The nucleic acid coding sequence for SEQ ID NO:1 is SEQ ID NO:2:

ATGGGGAACACCATCAGGGCCCTCGTGGCCTTCATCCCTGCTGAC
CGTTGCCAGAACTATGTGGTCAGGGACCTCCGTGAGATGCCGCTG
GACAAGATGGTGGATCTGAGTGGGAGCCAGTTACGCCGCTTCCCC
CTGCACGTGTGCTCCTTCAGGGAGCTGGTCAAGCTCTACCTGAGC
GACAACCACCTCAATAGCCTGCCTCCGGAGCTGGGGCAGCTACAG
AACCTGCAGATTCTGGCCTTGGATTTCAACAACTTCAAGGCTCTG
CCCCAGGTGGTGTGCACCTTGAAACAGCTCTGCATCCTCTACCTG
GGCAACAACAAACTCTGCGACCTCCCCAGTGAGCTGAGCCTGCTC
CAGAACCTCAGGACCCTGTGGATCGAGGCCAACTGCCTCACCCAG
CTGCCGGATGTGGTCTGTGAGCTGAGTCTCCTTAAGACTCTGCAT
GCCGGCTCCAACGCCCTGCGTTTGCTGCCAGGCCAGCTCCGGCGC
CTCCAGGAGCTGAGGACCATCTGGCTCTCGGGCAACCGGCTAACT
GACTTTCCCACTGTGCTGCTTCACATGCCCTTCCTGGAGGTGATT
GATGTGGACTGGAACAGCATCCGTTACTTCCCCAGCCTGGCGCAC
CTGTCAAGTCTGAAGCTGGTCATCTATGACCACAATCCTTGCAGG
AACGCACCCAAGGTGGCCAAAGGTGTGCGCCGTGTGGGGAGATGG
GCAGAGGAGACGCCAGAGCCCGACCCTAGAAAAGCCAGGCGCTAT
GCGTTGGTCAGAGAGGAAAGCCAGGAGCTACAGGCACCAGTCCCT
CTACTTCCTCCTACCAACTCCTGA

In various versions of the invention, the LRRC10 protein comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:1.

“Ectopically introducing” (or grammatical variants thereof such as “ectopic introduction” or “ectopically introduce”) as used herein refers to ectopically expressing the LRRC10 protein in the cardiac cell or ectopically delivering the LRRC10 protein in the cardiac cell.

“Ectopically expressing” (or grammatical variants thereof such as “ectopic expression” or “ectopically express”) as used herein refers to inducing expression of a protein in a cell with an ectopic agent.

“Ectopic agent” as used herein refers to an agent that is not natively produced by a given cell and/or does not originate in a given cell (i.e., is originally produced outside the cell and is subsequently delivered within the cell). An exemplary ectopic agent is an ectopic nucleic acid. In some versions, ectopically expressing the LRRC10 protein comprises expressing the LRRC10 protein from an ectopic nucleic acid encoding the LRRC10 protein.

“Ectopic nucleic acid” as used herein refers to a nucleic acid that is a recombinant nucleic acid, an exogenous nucleic acid, or both.

“Recombinant nucleic acid” as used herein refers to a nucleic acid comprising a non-naturally occurring sequence. Non-limiting examples of recombinant nucleic acids include chromosomal or non-chromosomal DNA (e.g., an extrachromosomal plasmid) comprising a recombinant gene, chromosomal DNA comprising a duplicate copy of a native gene inserted in a non-native locus, chromosomal DNA or non-chromosomal DNA (e.g., an extrachromosomal plasmid) comprising a native or non-native coding sequence operatively linked to a heterologous genetic element (such as a promoter, enhancer, etc.), viral RNA, messenger RNA (mRNA) comprising at least one base difference with respect to the cell's native mRNA, and nucleoside-modified messenger RNA (modRNA), among others. In some versions of the invention, the ectopic expression comprises expressing the LRRC10 protein from a recombinant nucleic acid encoding the LRRC10 protein within the cardiac cell. In some versions, the recombinant nucleic acid is also an exogenous nucleic acid.

“Exogenous nucleic acid” as used herein refers to a nucleic acid that does not originate within a given cell, i.e., is originally produced outside the cell and is subsequently delivered within the cell. Non-limiting examples of exogenous nucleic acids include DNA or RNA delivered to a cell with a transduction reagent (e.g., DNA or RNA viruses) and DNA or RNA delivered to a cell with a transfection reagent (e.g., liposomes, cationic polymer, or dendrimer nanoparticle), among others. In some versions of the invention, the exogenous expression comprises expressing the LRRC10 protein from an exogenous nucleic acid encoding the LRRC10 protein within the cardiac cell. In some versions, the methods comprise delivering the exogenous nucleic acid within the cardiac tissue cell. In some versions, the exogenous nucleic acid is also a recombinant nucleic acid.

In some versions, the ectopic nucleic acid comprises a gene encoding the LRRC10 protein. In some versions, the gene is an ectopic gene. In some versions, the gene is a recombinant gene. In some versions, the gene is a exogenous gene. In some versions, the gene is a recombinant exogenous gene. In some versions, the gene comprises a heart-specific promoter. “Heart-specific promoter” refers to a promoter that is exclusively or primarily active in cardiac cells. Examples of heart-specific promoters include the myosin light chain (MLC2v) promoter (Phillips et al. 2002, Boecker et al. 2004), the α-myosin heavy chain (α-MHC) promoter (Lee et al. 2011), the troponin 2 (TNTT2) promoter (Fiedorowicz et al. 2020), the alpha cardiac actin (ACTC) promoter (Fiedorowicz et al. 2020), the cardiac troponin-T (cTnT) promoter (Lee et al. 2011), and the following examples, among others. In some versions, the ectopic nucleic acid comprises an mRNA encoding the LRRC10 protein. In some versions, the mRNA is an ectopic mRNA. In some versions, the mRNA is a recombinant mRNA. In some versions, the mRNA is a exogenous mRNA. In some versions, the mRNA is a recombinant exogenous mRNA. In some versions, the mRNA is a “nucleoside-modified” or “modified” mRNA (modRNA). “Ectopic” used with respect to a gene or mRNA refers to a gene or mRNA that is recombinant, exogenous, or recombinant and exogenous. “Recombinant” used with respect to a gene or mRNA refers to a gene or mRNA comprising a non-naturally occurring sequence. “Exogenous” as used herein with respect to a gene or mRNA refers to a gene or mRNA that does not originate within a given cell, i.e., is originally produced outside the cell and is subsequently delivered within the cell.

“Expression” as used herein refers to the production of a protein from one or more nucleic acids. Examples include translation of an mRNA; transcription of a DNA to an mRNA followed by translation of the mRNA; and reverse transcription of an RNA to a cDNA, transcription of the cDNA to an mRNA, and translation of the mRNA to a protein. Expression of a protein is considered herein to be ectopic if any one or more nucleic acids involved in the production of the protein is an ectopic nucleic acid. In some versions, the ectopic expression comprises overexpression. “Overexpression” refers to expression of a protein in a given cell above a native level of expression.

“Ectopically delivering” as used herein refers to delivering (transferring) a pre-existing LRRC10 protein from outside a cell to inside the cardiac cell. An ectopically delivered protein is not expressed within the cell. The ectopically delivered LRRC10 protein can be a recombinant form of the native LRRC10 protein or have a native LRRC10 sequence. Methods for ectopically delivering proteins to cells are described by Yu et al. 2021, among others.

Ectopic agents also include small molecules, transcription factors, antibodies (e.g., intrabodies), that can induce expression (such as overexpression) of LRRC10 in a cell.

The cardiac cell in which the LRRC10 protein is expressed is preferably a mammalian cardiac cell, such as a human cell. The cardiac cell is preferably a cardiomyocyte.

“Cardiac cell” as used herein can refer to a single cardiac cell or a plurality of cardiac cells. In some versions, the LRRC10 protein is ectopically expressed in the plurality of cardiac cells.

In some versions, the cardiac cell in which the LRRC10 protein is expressed is comprised within cardiac tissue. “Cardiac tissue” as used herein refers to tissue comprising one or more types of cardiac cells (e.g., cardiomyocytes, fibroblasts, endothelial cells, and perivascular cells). The cardiac tissue can be incorporated in a whole heart, isolated from a whole heart, or generated at least in part in vitro. In some versions, the LRRC10 protein is ectopically expressed in the cardiac tissue cells.

In some versions, the cardiac cell in which the LRRC10 protein is expressed is comprised within a heart. The heart can be comprised within a subject, isolated from a subject, or generated at least in part in vitro. In some versions, the LRRC10 protein is expressed in the cells of the heart.

In some versions, the heart comprising the cardiac cell in which the LRRC10 protein is expressed comprises a cardiac injury. “Cardiac injury” as used herein refers to any structural cardiac damage or defect and/or any functional defect. In some versions, the cardiac injury results from a cardiac event. In some versions, the cardiac injury is a congenital defect.

Cardiac structural damage is referred to herein as a “myocardial lesion” or “cardiac lesion.” Nonlimiting examples of myocardial lesions include myocardial infarcts, decreased myocardial thickness, fibrosis, dilated cardiac chamber, increased left ventricular end-diastolic diameter, increased left ventricular end-systolic diameter, and combinations thereof. A decrease in myocardial thickness is understood to be determined relative to surrounding areas of the heart tissue. “Increased” in the context of increased left ventricular end-diastolic diameter and increased left ventricular end-systolic diameter refers to values above normal values. Ranges for normal (non-defective) values for left ventricular end-diastolic diameter and left ventricular end-systolic diameter are known in the art.

“Functional defect” as used herein refers to any type of reduced heart function, by any amount, with respect to a normal functioning heart. Examples of functional defects, for example, include ejection fraction defects, fractional shortening defects, end-systolic elastance defects, and combinations thereof. Ejection fraction defects include reduced ejection fraction. Fractional shortening defects include reduced fractional shortening. End-systolic elastance defects include reduced end-systolic elastance. “Reduced” and “increased” in these contexts refer to values below and above normal values, respectively. Ranges for normal (non-defective) values for ejection fraction, fractional shortening, end-systolic elastance are known in the art.

In some versions, the LRRC10 protein is expressed in an amount and for a time effective to elicit an amelioration of the cardiac injury. “Ameliorate” in this context refers to any improvement of the cardiac injury. Accordingly, in some versions, the LRRC10 protein is expressed in an amount and for a time effective to elicit an improvement in cardiac structure, such as a decrease in myocardial lesion size, an increase in myocardial thickness, decreased fibrosis, a decrease in left ventricular end-diastolic diameter, a decrease in left ventricular end-systolic diameter, an increase in coronary artery formation, an increase in capillary density, an increase in revascularization, or any combination thereof. In some versions, the LRRC10 protein is expressed in an amount and for a time effective to elicit an improvement in cardiac function, such as an increase in an increase in ejection fraction, an increase in fractional shortening, an increase in end-systolic elastance, or any combination thereof.

In some versions, the cardiac cell in which the LRRC10 protein is expressed is comprised within a subject, such as within cardiac tissue within a subject, such as within a heart of a subject. The subject is preferably a mammal, such as a human.

In some versions, the subject is a subject who comprises a cardiac injury. The cardiac injury can comprise any cardiac injury, including any of those explicitly described herein. In some versions, the cardiac injury is a myocardial infarct.

In some versions, the subject is suffering from or has suffered from a cardiac event or chronic heart condition. “Cardiac event” as used herein refers to any acute event causing or having the potential to cause a cardiac injury. Nonlimiting examples of cardiac events include cardiac ischemia, cardiac ischemia-reperfusion, myocardial infarction, myocarditis, and blunt trauma. “Chronic heart condition” as used herein refers to a persisting or recurring condition causing or having the potential to cause a cardiac injury. Nonlimiting examples of chronic heart conditions include coronary heart disease, valve diseases, high blood pressure, congenital heart conditions, and inherited heart conditions. In some versions, the subject is suffering from or has suffered from heart failure. In some versions, the heart failure comprises heart failure with reduced ejection fraction (dilated cardiomyopathy).

Some versions of the invention comprise administering a nucleic-acid delivery reagent comprising an ectopic nucleic acid to the subject. The ectopic nucleic acid preferably comprises a sequence encoding an LRRC10 protein. “Nucleic-acid delivery reagent” as used herein refers to any reagent capable of being administered to the subject and delivering the ectopic nucleic acid in the cardiac cell. Reagents suitable for this purpose are well-known in the art. These include transduction reagents and transfection reagents. “Transduction reagent” as used herein refers to a reagent capable of delivering a nucleic acid into a cell via viral infection. Examples of transfection reagents include DNA or RNA viral vectors, such as adenoviruses, adeno-associated viruses, herpes simplex viruses, and retroviruses (e.g., lentiviruses), among others. “Transfection reagent” as used herein refers to a reagent capable of delivering a nucleic acid into a cell via non-viral methods. Examples of transfection reagents include liposomes, cationic polymers, and dendrimer nanoparticles, among others. In some versions, the nucleic-acid delivery reagent is simply a naked nucleic acid, which can be delivered into a heart cell via electroporation or other mechanisms.

In some versions, the ectopic nucleic acid in the nucleic-acid delivery reagent is a DNA, such as a DNA construct or plasmid DNA. In some versions, the DNA in the nucleic-acid delivery reagent comprises a gene, such as an ectopic gene, which may be a recombinant gene. In some versions, the gene comprises a heart-specific promoter. In some versions, the ectopic nucleic acid in the nucleic-acid delivery reagent is an RNA. In some versions, the ectopic nucleic acid in the nucleic-acid delivery reagent is a messenger RNA (mRNA). In some versions, the ectopic nucleic acid in the nucleic-acid delivery reagent is a “nucleoside-modified” or “modified” mRNA (modRNA). modRNAs are well known in the art of gene therapy. See, e.g., Goswami et al. 2019, Magadum et al. 2018, Zangi et al. 2021, Korpela et al. 2021., US 2016/0250290 A1, U.S. Pat. No. 7,256,182 B2, US 2008/0200661 A1, and the references cited therein for examples of nucleic-acid delivery reagent reagents, such as transduction and transfection reagents, suitable for delivering ectopic nucleic acids to heart cells, such as cardiomyocytes.

The nucleic-acid delivery reagent can be administered to the subject via injection, infusion, oral administration, or any other suitable mode of administration.

The delivered ectopic nucleic acid can be maintained in the cell extra-chromosomally or be incorporated within one of the cell's chromosomes.

Another aspect of the invention is directed to methods of inducing cardiac regeneration in a mammalian subject suffering from cardiac injury. The method comprises ectopically expressing an LRRC10 protein in a cardiac cell in the subject to thereby induce cardiac regeneration in the subject. “Cardiac regeneration” as used herein refers to any improvement of a cardiac injury, whether structurally, functionally, or both. In some versions of the invention, the cardiac regeneration comprises increased proliferation of cardiomyocytes. In some versions of the invention, the cardiac regeneration comprises increased contractility of cardiomyocytes. Various other aspects of cardiac regeneration may depend on the type of cardiac injury sustained by the subject. Any aspects described above with respect to inducing cardiac cell proliferation can apply to the methods of inducing cardiac regeneration.

In some versions, the LRRC10 protein is ectopically introduced to a subject that has deficient LRRC10 expression, such as an adult mammal, or an adult human. “Deficient LRRC10 expression” refers to a lower level of expression in a cell with respect to a level of expression in a cell from a neonatal subject, such as a neonatal mammal or a neonatal human.

Unless otherwise indicated, the accession numbers referenced herein are derived from the NCBI database (National Center for Biotechnology Information) maintained by the National Institute of Health, U.S.A.

The term “alignment” refers to a method of comparing two or more nucleic acid or protein sequences for the purpose of determining their relationship to each other. Alignments are typically performed by computer programs that apply various algorithms, however it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW, (see, Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22: 4673-4680, 1994); CLUSTALV, (see, Larkin M. A., et al., CLUSTALW2, ClustalW and ClustalX version 2, Bioinformatics 23(21): 2947-2948, 2007); Jotun-Hein, Muscle et al., MUSCLE: a multiple sequence alignment method with reduced time and space complexity, BMC Bioinformatics 5: 113, 2004); Mafft, Kalign, ProbCons, and T-Coffee (see Notredame et al., T-Coffee: A novel method for multiple sequence alignments, Journal of Molecular Biology 302: 205-217, 2000). Exemplary programs that implement one or more of the above algorithms include, but are not limited to MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif. 92121). In a non-limiting example, MegAlign is used to implement the CLUSTALW alignment algorithm with the following parameters: Gap Penalty 10, Gap Length Penalty 0.20, Delay Divergent Seqs (30%) DNA Transition Weight 0.50, Protein Weight matrix Gonnet Series, DNA Weight Matrix IUB.

The term “consensus sequence” or “canonical sequence” refers to an archetypical amino acid sequence against which all variants of a particular protein or sequence of interest are compared. Either term also refers to a sequence that sets forth the nucleotides that are most often present in a nucleic acid sequence of interest. For each position of a protein, the consensus sequence gives the amino acid that is most abundant in that position in the sequence alignment.

The term “conservative substitutions” or “conserved substitutions” refers to, for example, a substitution of an amino acid with a conservative variant.

“Conservative variant” refers to residues that are functionally similar to a given residue. Amino acids within the following groups are conservative variants of one another: glycine, alanine, serine, and proline (very small); alanine, isoleucine, leucine, methionine, phenylalanine, valine, proline, and glycine (hydrophobic); alanine, valine, leucine, isoleucine, methionine (aliphatic-like); cysteine, serine, threonine, asparagine, tyrosine, and glutamine (polar); phenylalanine, tryptophan, tyrosine (aromatic); lysine, arginine, and histidine (basic); aspartate and glutamate (acidic); alanine and glycine; asparagine and glutamine; arginine and lysine; isoleucine, leucine, methionine, and valine; and serine and threonine.

“Corresponding proteins” as used herein refers to first and second proteins that are identical in sequence except for a given set of modifications.

The terms “corresponds to” and “corresponding to” used with reference to an amino acid residue or position refer to an amino acid residue or position in a first protein sequence being positionally equivalent to an amino acid residue or position in a second reference protein sequence by virtue of the fact that the residue or position in the first protein sequence aligns to the residue or position in the reference sequence using bioinformatic techniques, for example, using the methods described herein for preparing a sequence alignment. The corresponding residue in the first protein sequence is then assigned the position number in the second reference protein sequence.

The term “deletion,” when used in the context of an amino acid sequence, means a deletion in or a removal of one or more residues from the amino acid sequence of a corresponding protein, resulting in a mutant protein having at least one less amino acid residue as compared to the corresponding protein. The term can also be used in the context of a nucleotide sequence, which means a deletion in or removal of a nucleotide from the nucleic acid sequence of a corresponding nucleic acid.

The term “DNA construct” is used herein to refer to a recombinant DNA that can be used, for example, to express a protein. Typically a DNA construct is generated in vitro by PCR or other suitable technique(s) known to those in the art. In certain embodiments, the DNA construct comprises a sequence of interest (e.g., an incoming sequence). In some embodiments, the sequence is operably linked to additional elements such as control elements (e.g., promoters, etc.). A DNA construct can further comprise a selectable marker. It can also comprise an incoming sequence flanked by homology targeting sequences. In a further embodiment, the DNA construct comprises other non-homologous sequences, added to the ends (e.g., stuffer sequences or flanks). In some embodiments, the ends of the incoming sequence are closed such that the DNA construct forms a closed circle. In some embodiments, the DNA construct comprises sequences homologous to the host cell chromosome. In other embodiments, the DNA construct comprises non-homologous sequences. Once the DNA construct is assembled in vitro it may be used to: 1) insert heterologous sequences into a desired target sequence of a host cell; 2) mutagenize a region of the host cell chromosome (i.e., replace an endogenous sequence with a heterologous sequence); 3) delete target genes; and/or (4) introduce a replicating plasmid into the host.

The term “expressed genes” refers to genes that are transcribed into messenger RNA (mRNA) and then translated into protein, as well as genes that are transcribed into other types of RNA, such as transfer RNA (tRNA), ribosomal RNA (rRNA), and regulatory RNA, which are not translated into protein.

The terms “expression cassette” or “expression vector” refer to a nucleic acid construct generated recombinantly or synthetically, with a series of specified elements that permit transcription of a particular nucleic acid in a target cell or in vitro. A recombinant expression cassette can be incorporated into a plasmid, chromosome, mitochondrial DNA, plasmid DNA, virus, or nucleic acid fragment. Typically, the recombinant expression cassette portion of an expression vector includes, among other sequences, a nucleic acid sequence to be transcribed and a promoter. In particular embodiments, expression vectors have the ability to incorporate and express heterologous nucleic acid fragments in a host cell. Many prokaryotic and eukaryotic expression vectors are commercially available. Selection of appropriate expression vectors is within the knowledge of those of skill in the art. The term “expression cassette” can be used interchangeably herein with “DNA construct,” and their grammatical equivalents.

“Gene” refers to a nucleic acid segment (e.g., a DNA segment) that encodes a protein, and may include regions preceding (e.g., promoter) and following the coding regions as well as intervening sequences (e.g., introns) between individual coding segments (e.g., exons).

The term “endogenous protein” refers to a protein that is native or naturally occurring. “Endogenous nucleic acid” refers to a naturally occurring nucleic acid that is in the cell and was not delivered into the cell.

The term “heterologous” used with reference to a protein or a nucleic acid in a host cell refers to a protein or a nucleic acid that does not naturally occur in the host cell.

The term “heterologous” used to describe two different amino acid or nucleic acid sequences refers to two sequences that are not naturally present together in the same protein or nucleic acid. The term “heterologous” used to describe two different protein domains refers to two protein domains that are not naturally present together in the same protein. As used herein, “domain” refers to any portion of protein that confers a particular structural and/or functional characteristic to a protein. Exemplary protein domains include signal peptides, extracellular domains, transmembrane domains, cytoplasmic domains, catalytic domains, affinity tags, and linkers, among others.

The term “homologous sequences” as used herein refers to a nucleic acid or protein sequence having, for example, about 100%, about 99% or more, about 98% or more, about 97% or more, about 96% or more, about 95% or more, about 94% or more, about 93% or more, about 92% or more, about 91% or more, about 90% or more, about 88% or more, about 85% or more, about 80% or more, about 75% or more, about 70% or more, about 65% or more, about 60% or more, about 55% or more, about 50% or more, about 45% or more, or about 40% or more sequence identity to another nucleic acid or protein sequence when optimally aligned for comparison. In particular embodiments, homologous sequences can retain the same type and/or level of a particular activity of interest. In some embodiments, homologous sequences have between 85% and 100% sequence identity, whereas in other embodiments there is between 90% and 100% sequence identity. In particular embodiments, there is 95% and 100% sequence identity.

“Homology” refers to sequence similarity or sequence identity. Homology is determined using standard techniques known in the art (see, e.g., Smith and Waterman, Adv. Appl. Math., 2:482, 1981; Needleman and Wunsch, J. Mol. Biol., 48:443, 1970; Pearson and Lipman, Proc. Natl. Acad. Sci. USA 85:2444, 1988; programs such as GAP, BESTFIT, FASTA, and TFASTA in the Wisconsin Genetics Software Package (Genetics Computer Group, Madison, Wis.); and Devereux et al., Nucl. Acid Res., 12:387-395, 1984). A non-limiting example includes the use of the BLAST program (Altschul et al., Gapped BLAST and PSI-BLAST: a new generation of protein database search programs, Nucleic Acids Res. 25:3389-3402, 1997) to identify sequences that can be said to be “homologous.” A recent version such as version 2.2.16, 2.2.17, 2.2.18, 2.2.19, or the latest version, including sub-programs such as blastp for protein-protein comparisons, blastn for nucleotide-nucleotide comparisons, tblastn for protein-nucleotide comparisons, or blastx for nucleotide-protein comparisons, and with parameters as follows: Maximum number of sequences returned 10,000 or 100,000; E-value (expectation value) of le-2 or le-5, word size 3, scoring matrix BLOSUM62, gap cost existence 11, gap cost extension 1, may be suitable. An E-value of le-5, for example, indicates that the chance of a homologous match occurring at random is about 1 in 10,000, thereby marking a high confidence of true homology.

The term “identical” (or “identity”), in the context of two nucleic acid or protein sequences, means that the residues in the two sequences are the same when aligned for maximum correspondence, as measured using a sequence comparison or analysis algorithm such as those described herein. For example, if when properly aligned, the corresponding segments of two sequences have identical residues at 5 positions out of 10, it is said that the two sequences have a 50% identity. Most bioinformatic programs report percent identity over aligned sequence regions, which are typically not the entire molecules. If an alignment is long enough and contains enough identical residues, an expectation value can be calculated, which indicates that the level of identity in the alignment is unlikely to occur by random chance.

The term “insertion,” when used in the context of an amino acid sequence, refers to an insertion of an amino acid with respect to the amino acid sequence of a corresponding protein, resulting in a mutant protein having an amino acid that is inserted between two existing contiguous amino acids, i.e., adjacent amino acids residues, which are present in the corresponding protein. The term “insertion,” when used in the context of a nucleic acid sequence, refers to an insertion of one or more nucleotides in the corresponding nucleic acid between two existing contiguous nucleotides, i.e., adjacent nucleotides, which are present in the corresponding nucleic acids.

The term “deliver” or “delivered” refers to, in the context of delivering a nucleic acid or protein into a cell, transferring the nucleic acid or protein into the cell. Methods for delivering a nucleic acid into a cell include but are not limited to protoplast fusion, transfection, transformation, conjugation, and transduction, among other methods described herein. Methods for delivering a protein into a cell are described by Yu et al. 2021, among others.

The term “isolated” or “purified” means a material that is removed from its original environment, for example, the natural environment if it is naturally occurring, or a cultivation broth if it is produced in a recombinant host cell cultivation medium. A material is said to be “purified” when it is present in a particular composition in a higher concentration than the concentration that exists prior to the purification step(s). For example, with respect to a composition normally found in a naturally occurring or wild type organism, such a composition is “purified” when the final composition does not include some material from the original matrix. As another example, where a composition is found in combination with other components in a recombinant host cell cultivation medium, that composition is purified when the cultivation medium is treated in a way to remove some component of the cultivation, for example, cell debris or other cultivation products, through, for example, centrifugation or distillation. As another example, a naturally occurring nucleic acid or protein present in a living animal is not isolated, but the same nucleic acid or protein, separated from some or all of the coexisting materials in the natural system, is isolated, whether such process is through genetic engineering or mechanical separation. Such nucleic acids can be parts of vectors. Alternatively, such nucleic acids or proteins can be parts of compositions. Such nucleic acids or proteins can be considered “isolated” because the vectors or compositions comprising thereof are not part of their natural environments. In another example, a nucleic acid or protein is said to be purified if it gives rise to essentially one band in an electrophoretic gel or a blot.

The term “mutation” refers to, in the context of a nucleic acid, a modification to the nucleic acid sequence resulting in a change in the sequence of a nucleic acid with reference to a corresponding nucleic acid sequence. A mutation to a nucleic acid sequence can be an alteration that does not change the encoded amino acid sequence, for example, with regard to codon optimization for expression purposes, or that modifies a codon in such a way as to result in a modification of the encoded amino acid sequence. Mutations can be introduced into a nucleic acid through any number of methods known to those of ordinary skill in the art, including random mutagenesis, site-specific mutagenesis, oligonucleotide directed mutagenesis, gene shuffling, directed evolution techniques, combinatorial mutagenesis, and site saturation mutagenesis, among others.

“Mutation” or “mutated” means, in the context of a protein or nucleic acid, a modification to the protein or nucleic acid sequence resulting in a change in the sequence with respect to a corresponding sequence. A mutation can refer to a substitution of one residue (amino acid or nucleotide) with another residue, an insertion of one or more residues, or a deletion of one or more residues. A mutation can include the replacement of a naturally occurring residue with a non-natural residue, or a chemical modification of a residue. A mutation can also be a truncation (e.g., a deletion or interruption) in a sequence or a subsequence with respect to a corresponding sequence. A “mutant” as used herein is a protein or nucleic acid comprising a mutation.

A “naturally occurring equivalent,” in the context of the present invention, refers to a naturally occurring version of a protein or nucleic acid, e.g., a naturally occurring protein or nucleic acid from which a recombinant protein or nucleic acid is derived.

The term “operably linked,” in the context of a nucleic acid sequence, refers to the placement of one nucleic acid sequence into a functional relationship with another nucleic acid sequence. A promoter or an enhancer is operably linked to a coding sequence if it affects the transcription of the sequence. A ribosome binding site is operably linked to a coding sequence if it is positioned so as to facilitate translation.

The term “optimal alignment” refers to the alignment giving the highest overall alignment score.

The terms “percent sequence identity,” “percent amino acid sequence identity,” “percent gene sequence identity,” and/or “percent nucleic acid sequence identity,” with respect to two proteins, nucleic acids and/or gene sequences (as appropriate), refer to the percentage of residues that are identical in the two sequences when the sequences are optimally aligned. Thus, 80% amino acid sequence identity means that 80% of the amino acids in two optimally aligned protein sequences are identical. The percent identities expressed herein with respect to a given named reference sequence are determined over the entire reference sequence, rather than only a portion thereof. Thus, a first RSV F sequence that is at least 80% identical to positions 26-494 of a second RSV F sequence, for example, is at least about 80% identical to the entire sequence of positions 26-494 of a second RSV F sequence, as opposed merely to subsequences thereof.

The term “plasmid” refers to a circular double-stranded (ds) DNA construct used as a cloning or expression vector, and which forms an extrachromosomal self-replicating genetic element in some eukaryotes or prokaryotes, or integrates into the host chromosome.

A “promoter” is a nucleic acid sequence that functions to direct transcription of a downstream gene. In preferred embodiments, the promoter is appropriate to the host cell in which the target gene is being expressed. The promoter, together with other transcriptional and translational regulatory nucleic acid sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

The terms “protein” and “polypeptide” are used interchangeably herein. The 3-letter code as well as the 1-letter code for amino acid residues as defined in conformity with the IUPAC-IUB Joint Commission on Biochemical Nomenclature (JCBN) is used throughout this disclosure. It is also understood that a protein may be coded for by more than one nucleic acid sequence due to the degeneracy of the genetic code. The terms “amino acid sequence,” “polypeptide sequence,” and “protein sequence” are used interchangeably herein.

The terms “nucleic acid” and “polynucleotide” are used interchangeably herein.

In general, the term “ectopic” used herein with respect to an element such as a nucleic acid, protein, gene, mRNA, promoter, vector, genetic element, etc. refers to an element that is recombinant, exogenous or both. “Recombinant” used herein with respect to such an element refers to an element comprising a non-naturally occurring sequence. “Exogenous” used herein with respect to such an element refers to an element that does not originate within a given cell, i.e., is originally produced outside the cell and is subsequently delivered within the cell.

The terms “regulatory segment,” “regulatory sequence,” or “expression control sequence” refer to a nucleic acid sequence that is operatively linked with another nucleic acid sequence that encodes the amino acid sequence of a polypeptide chain to effect the expression of that encoded amino acid sequence. The regulatory sequence can inhibit, repress, promote, or even drive the expression of the operably-linked nucleic acid sequence encoding the amino acid sequence.

The term “substantially identical,” in the context of two nucleic acids or two proteins refers to a nucleic acid or protein that comprises at least 70% sequence identity, for example, at least 75%, at least 80%, at least 85%, at least 90%, at least 91%, at least 92%, at least 93%, at least 94%, at least 95%, at least 96%, at least 97%, at least 98%, or at least 99% sequence identity as compared to a reference sequence using the programs or algorithms (e.g., BLAST, ALIGN, CLUSTAL) using standard parameters.

“Substantially purified” means molecules that are at least about 60% free, preferably at least about 75% free, about 80% free, about 85% free, and more preferably at least about 90% free from other components with which they are naturally associated. As used herein, the term “purified” or “to purify” also refers to the removal of contaminants from a sample.

“Substitution” means replacing an amino acid in the sequence of a corresponding protein with another amino acid at a particular position, resulting in a mutant of the corresponding protein. The amino acid used as a substitute can be a naturally occurring amino acid, or can be a synthetic or non-naturally occurring amino acid.

“Vector” refers to a nucleic acid construct or reagent designed to deliver nucleic acids into one or more cell types. Vectors include cloning vectors, expression vectors, shuttle vectors, plasmids, viruses, cassettes and the like.

“Wild type” means, in the context of gene or protein, a nucleic acid or protein with a sequence as it occurs in nature. In some embodiments, the wild-type sequence refers to a sequence of interest that is a starting point for protein engineering. “Wild type” is used interchangeably with “native.”

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### LRRC10 Regulates Mammalian Cardiomyocyte Cell Cycle During Heart Regeneration

### Summary

Leucine-rich repeat containing 10 (LRRC10) is a cardiomyocyte-specific protein, but its role in cardiac biology is little understood. In this study, we demonstrate that Lrrc10^{-/-} knockout mice exhibit a loss of the neonatal mouse regenerative response marked by reduced cardiomyocyte cytokinesis and increased cardiomyocyte binucleation. Interestingly, LRRC10 deletion disrupts the regenerative transcriptional landscape of the regenerating neonatal mouse heart. Remarkably, cardiac overexpression of LRRC10 restores cardiomyocyte cytokinesis, increases cardiomyocyte mononucleation, and the cardiac regenerative capacity of Lrrc10^{-/-} mice. Our results are consistent with a model in which LRRC10 is required for cardiomyocyte cytokinesis as well as regulation of the transcriptional landscape during mammalian heart regeneration.

### Results

The adult human heart shows little regenerative capacity following an ischemic injury, such as a myocardial infarction (MI), in which contractile cardiac muscle is replaced with a fibrotic scar (Murphy et al. 2020). In contrast, some species of fish and amphibians exhibit the capability of adult heart regeneration in response to injury (Becker et al. 1974, Cano-Martinez et al. 2010, Poss et al. 2002). In addition, neonatal mice and neonatal pigs exhibit a transient potential to undergo heart regeneration after an induced MI (Porrello et al. 2011, Zhu et al. 2018, Ye et al. 2018). New understanding of the mechanisms underlying cardiac regeneration provides opportunities for innovative cardiac regenerative therapies following MI to prevent the progression to heart failure and increased risk of early death (Sadek et al. 2020).

LRRC10 is a cardiomyocyte-specific member of the leucine-rich repeat (LRR) motif family of proteins that mediate protein-protein interactions (Kobe et al. 1994). LRRC10 knockout (Lrrc10^{-/-}) mice exhibit mild systolic dysfunction, first detected at embryonic day (E)17.5 without changes in cardiac structure or evidence of fibrosis, and postnatally, the Lrrc10^{-/-} mice gradually develop a dilated cardiomyopathy but exhibit normal survival (Brody et al. 2012). Interestingly, LRRC10 has been recently demonstrated to regulate Caᵥ1.2 channel function and contribute to homeostasis of intracellular Ca²⁺ cycling (Woon et al. 2018). In this study, we demonstrate that Lrrc10 deletion blocks neonatal heart regeneration. Remarkably, Lrrc10 deletion did not disrupt nuclear division (karyokinesis) during the early stages of M-phase of cardiomyocyte cell cycle, but specifically reduces cardiomyocyte cytoplasmic division (cytokinesis), which results in increased cardiomyocyte binucleation. Transcriptional analysis revealed a unique signature following Lrrc10 deletion compared to the regenerating control hearts. Importantly, LRRC10 overexpression during neonatal MI rescued the cardiac regenerative capacity of Lrrc10^{-/-} mice, demonstrating an important role for LRRC10 in regulating cardiomyocyte cytokinesis and cardiac regeneration. Our results reveal a key role for LRRC10 in postnatal cardiomyocyte cell cycle exit and mammalian cardiac regenerative potential.

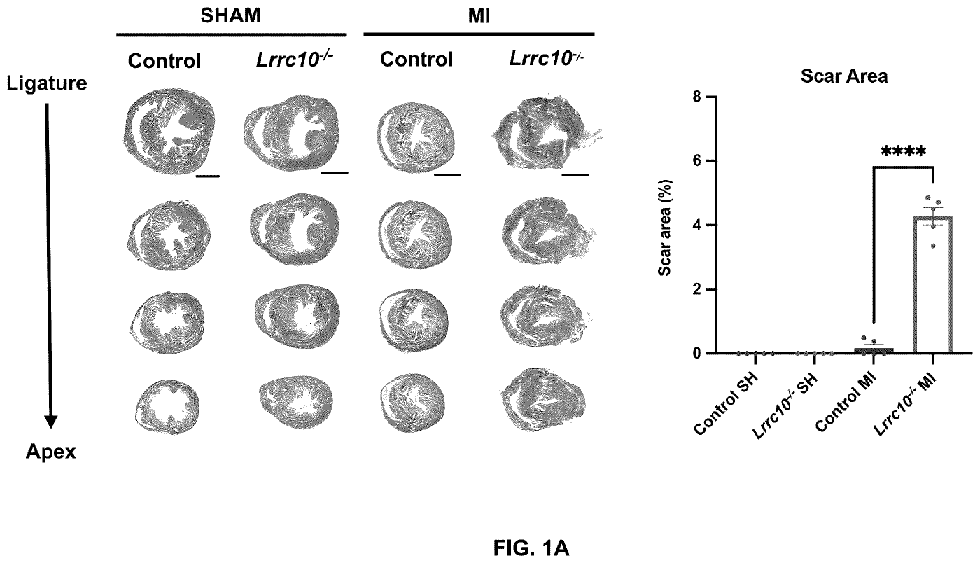

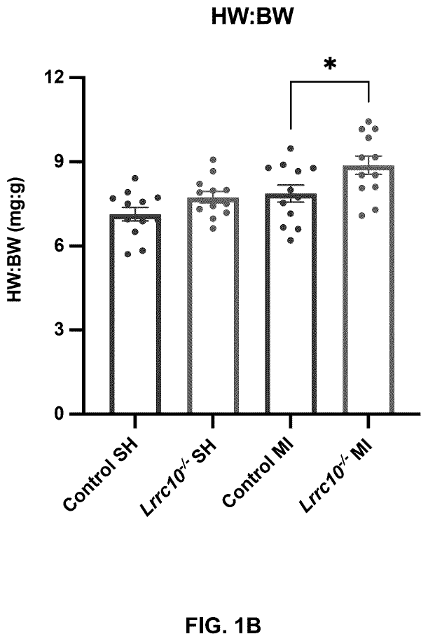

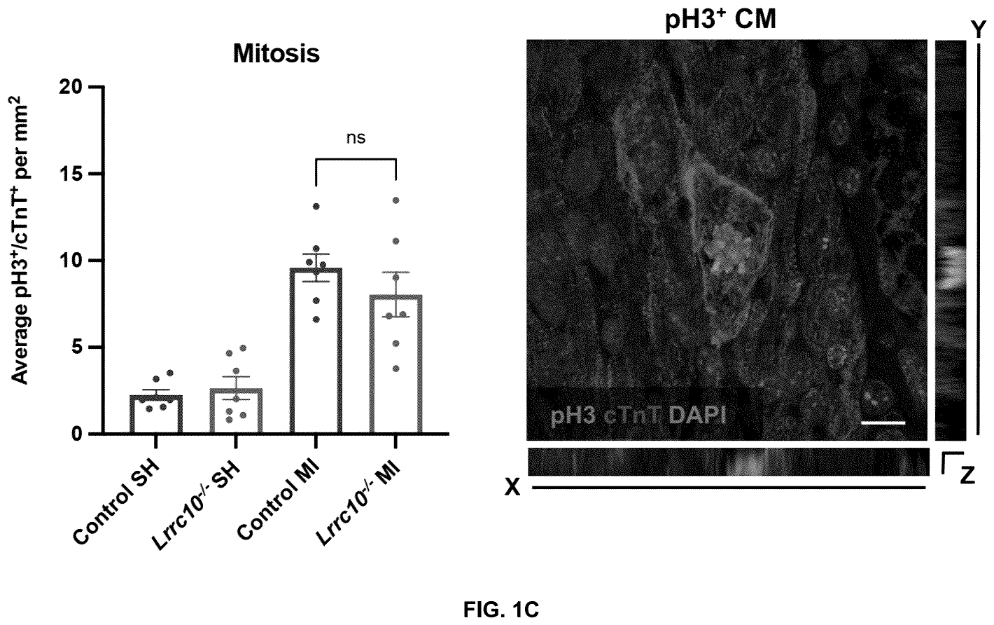

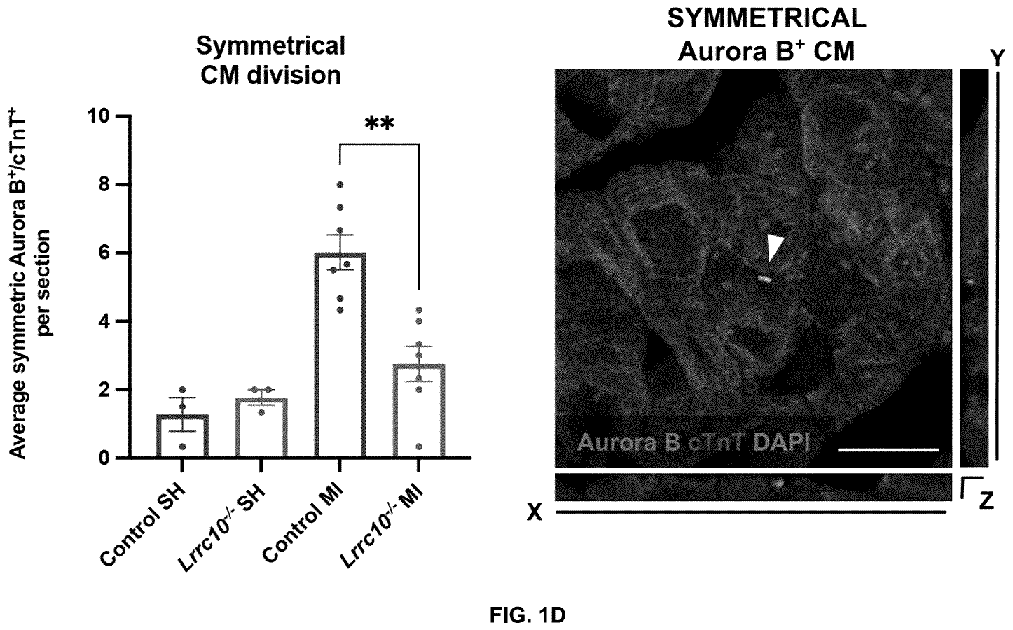

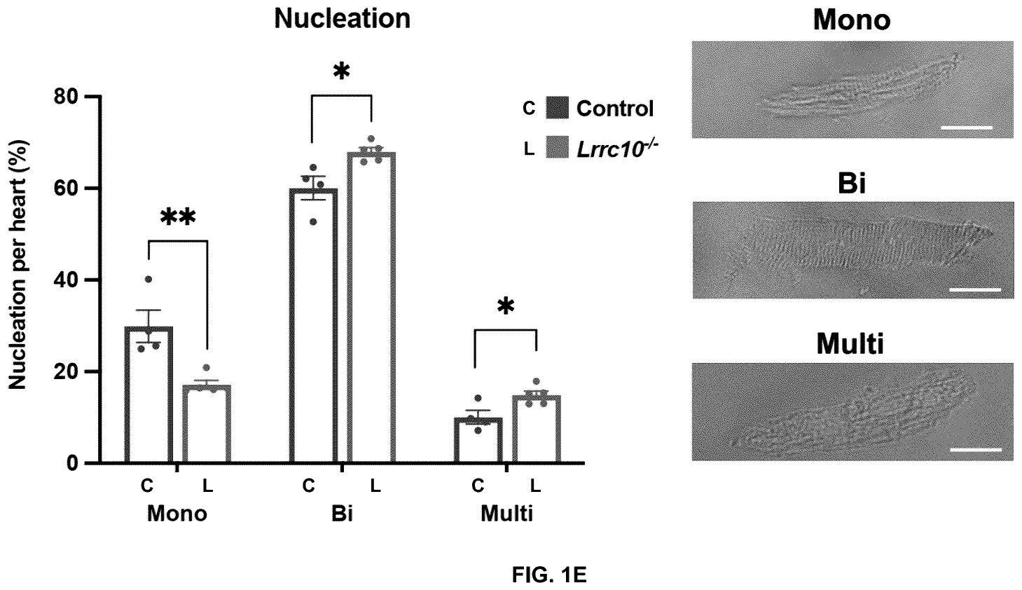

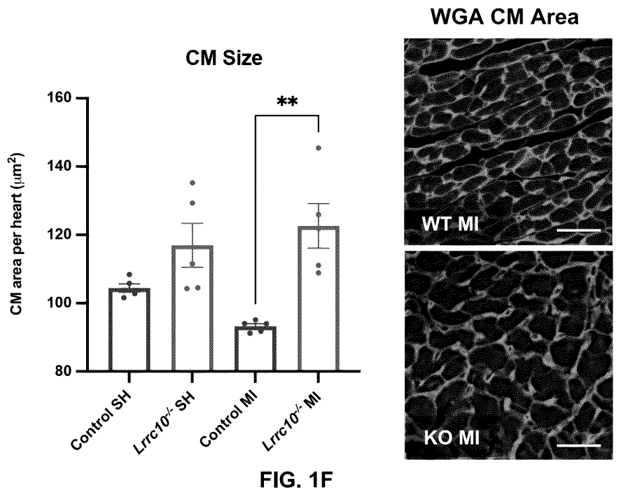

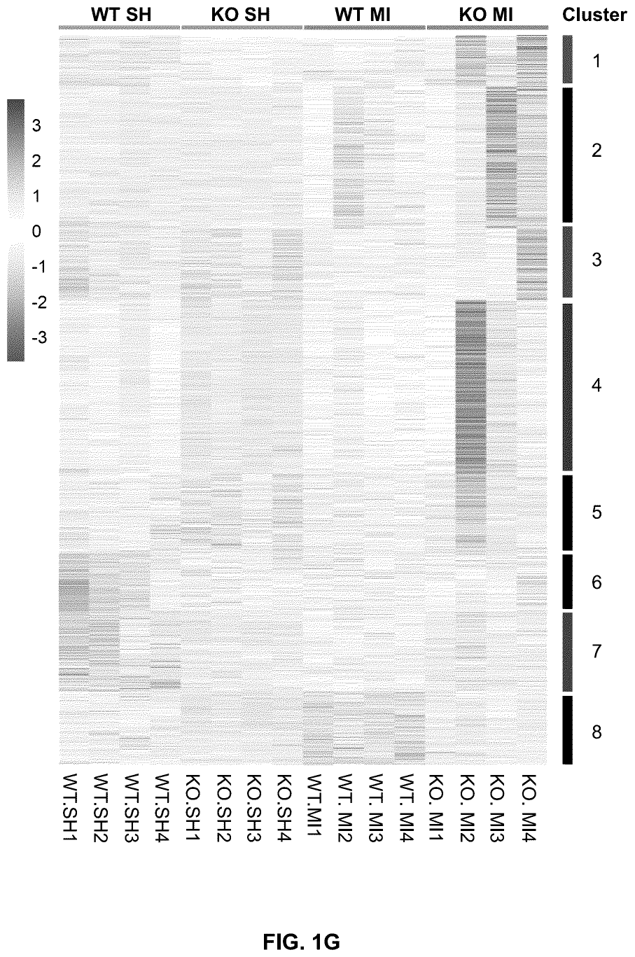

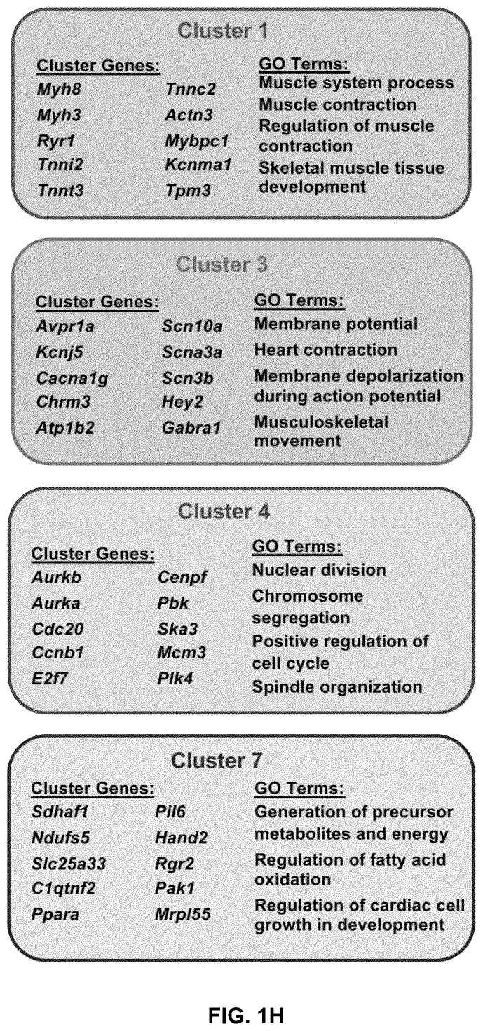

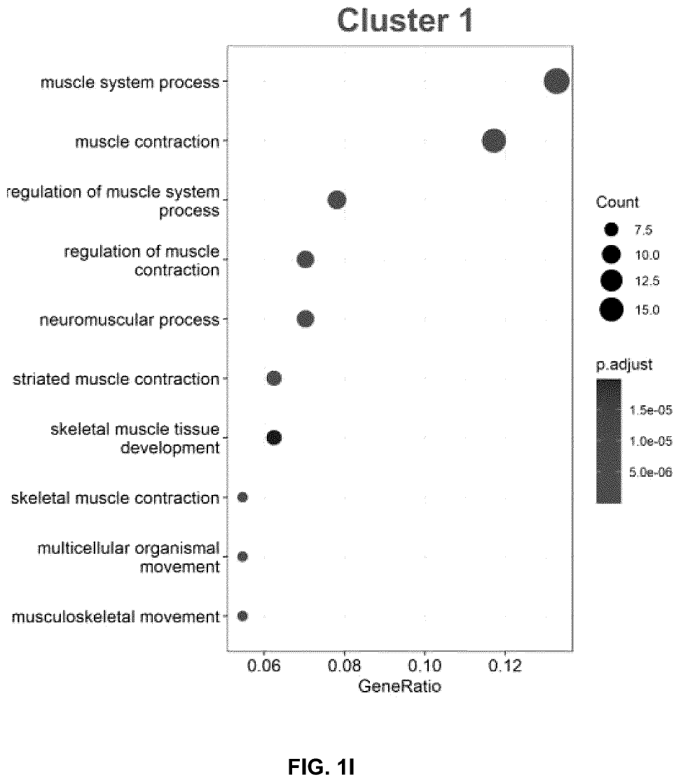

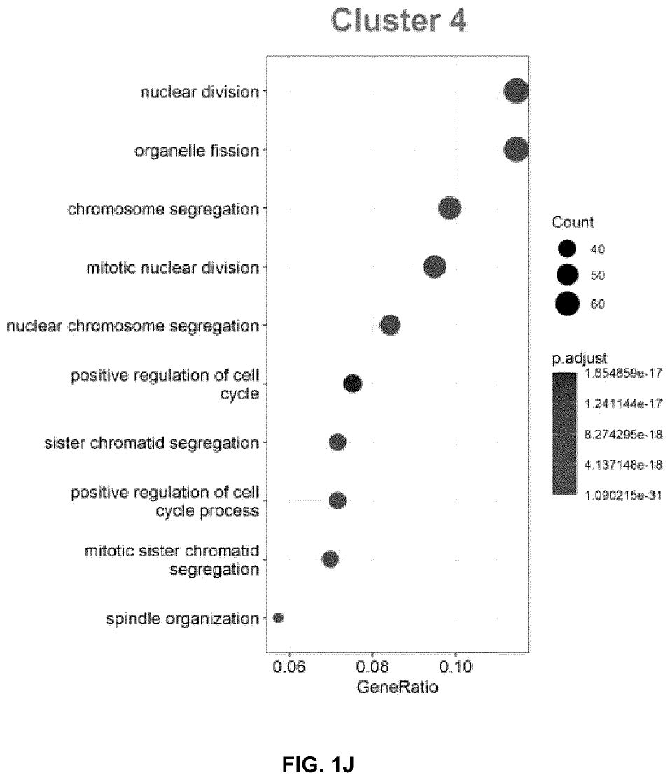

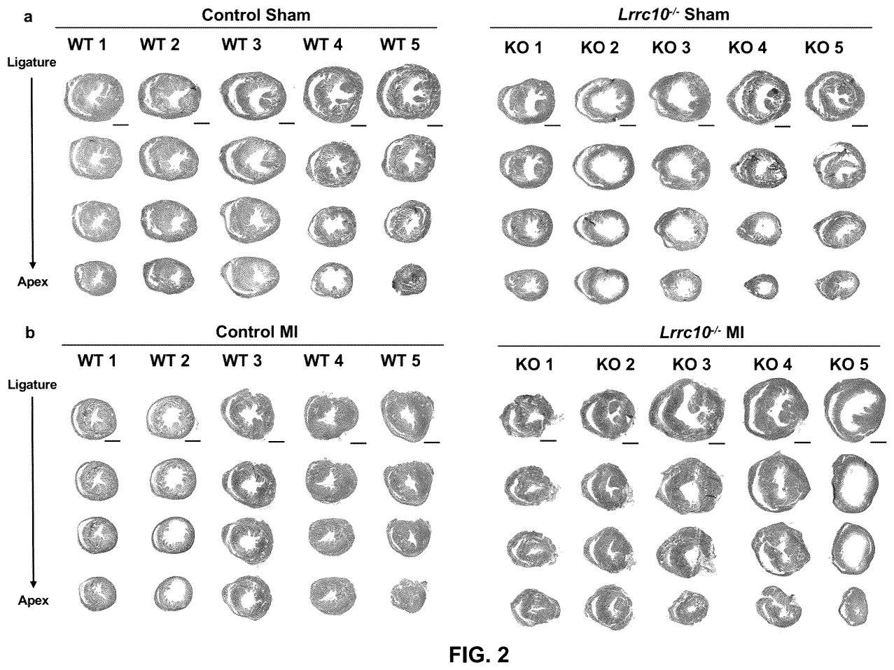

The role of LRRC10 in mammalian heart regeneration has not been explored. Lrrc10^{-/-} mice postnatally develop a slowly progressive dilated cardiomyopathy (Brody et al. 2012), an indication that LRRC10 is critical for cardiac homeostasis. To determine whether LRRC10 plays a role in mammalian heart regeneration, we evaluated the regenerative capacity of the Lrrc10^{-/-} mouse heart following an MI in postnatal-day 1 (P1) mice (Mahmoud et al. 2014). We utilized the whole-body Lrrc10 knockout mouse model, Lrrc10^{-/-}, as LRRC10 is a cardiomyocyte-specific protein (Adameyko et al. 2005, Kim and Antkiewicz et al. 2007, Kim and Kim et al. 2007). To determine the effect of Lrrc10 deletion on myocardial regeneration, we performed Masson's trichrome staining at 21 days post-MI in wild type (WT) control and Lrrc10^{-/-} mice. Remarkably, Lrrc10^{-/-} mice showed increased scar size and incomplete myocardial regeneration in comparison to control hearts (FIGS. 1A and 2). Since LRRC10 is a cardiomyocyte-specific protein, this increase in fibrosis is likely due to disruption of the cardiac regenerative response at the cardiomyocyte level rather than an impact on fibroblasts. In addition, we quantified a significant increase in heart weight to body weight ratio in Lrrc10^{-/-} mice post-MI (FIG. 1B). At 21-days post-sham surgery, no difference in heart weight: body weight was identified between WT and Lrrc10^{-/-} mice (FIGS. 1A, B, and 2), indicating there has been no significant remodeling in Lrrc10^{-/-} hearts by this developmental timepoint. These results demonstrate an evolutionarily conserved role for LRRC10 in regulating cardiac regeneration.

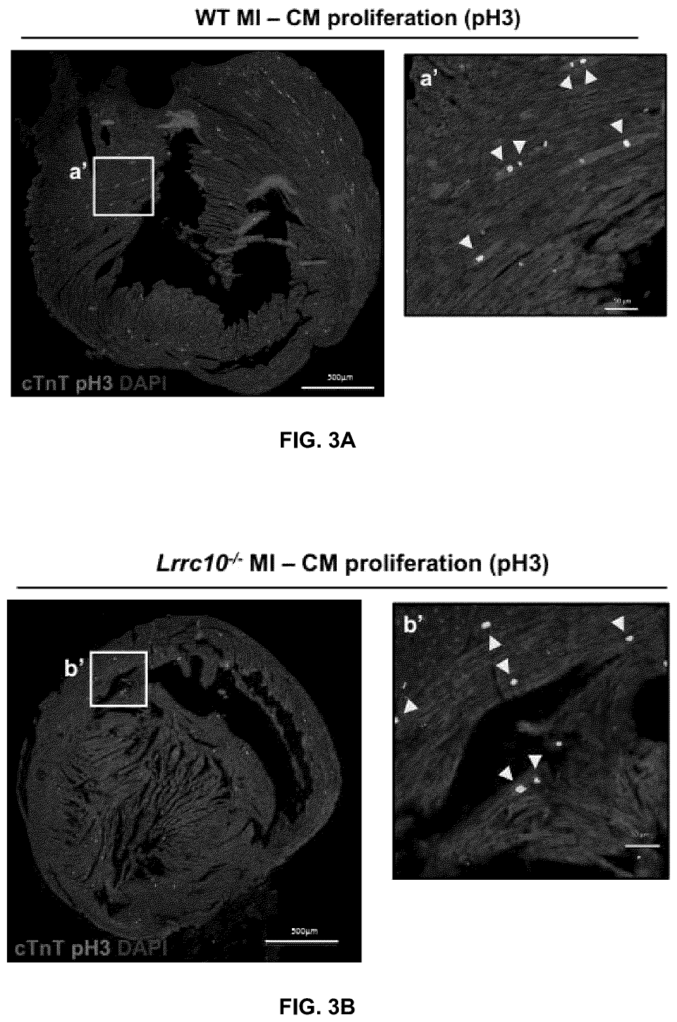

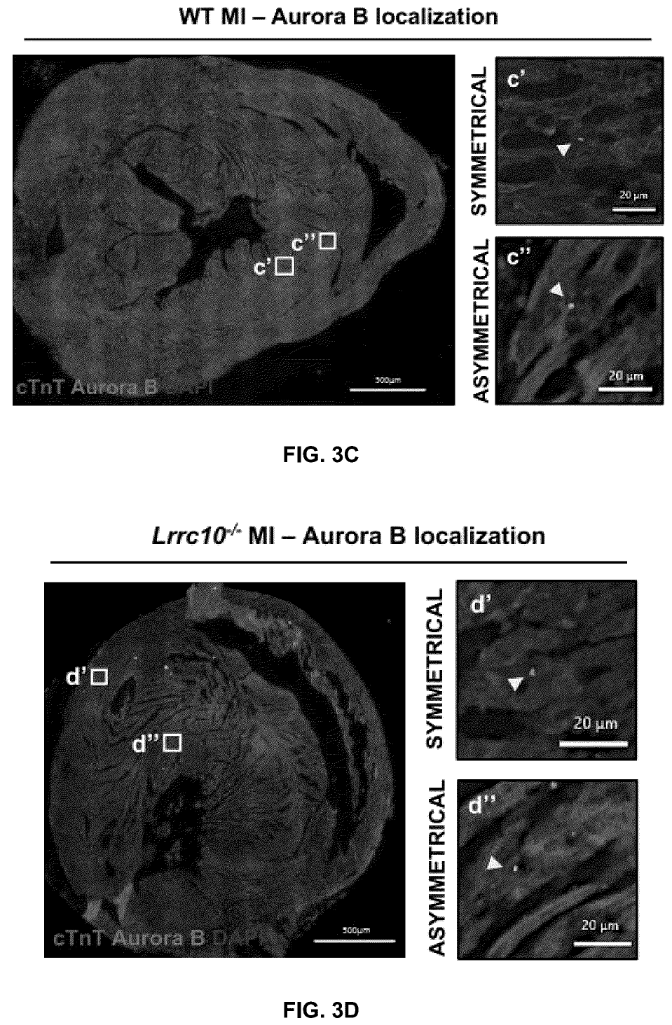

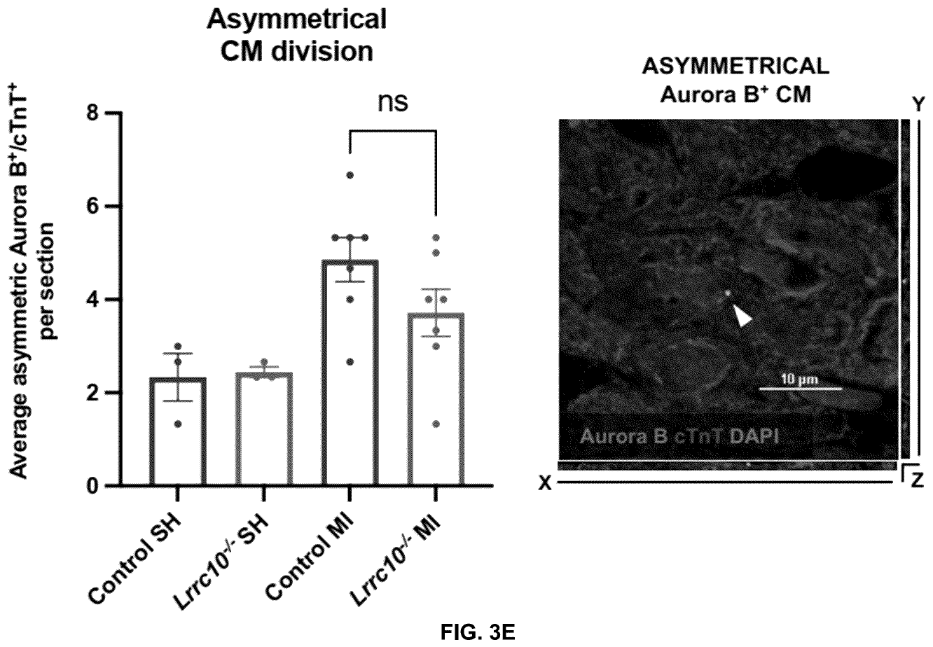

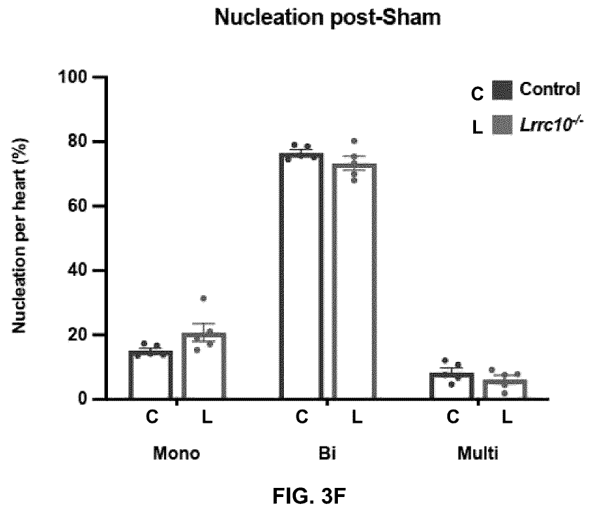

Heart regeneration is mediated by the proliferation of the pre-existing cardiomyocytes (Porrello et al. 2011). Here, we analyze two stages of M-phase during cardiomyocyte cell cycle. We use phospho-Histone3 (pH3) as a marker for early G2-M phase to measure karyokinesis. In addition, we use Aurora B as a marker of cytokinesis, which is symmetrically localized at the cleavage furrow to measure cytoplasmic division. During mammalian heart regeneration, cardiomyocyte proliferation peaks around 7 days post-MI (Porrello et al. 2011, Porrello et al. 2013). To establish the impact of Lrrc10 deletion on cardiomyocyte proliferation and division, we performed immunostaining for the early M-phase marker pH3 and the cardiomyocyte marker cardiac troponin T (cTnT) at 7 days following MI at P1. We found no significant difference in levels of pH3 positive cardiomyocytes between WT control and Lrrc10^{-/-} mice (FIGS. 1C, 3A, and 3B). Our results are similar to the prior cavefish and zebrafish study, which showed comparable levels of induced DNA synthesis in both controls and Lrrc10^{-/-} zebrafish at the same timepoint (Stockdale et al. 2018). However, mammalian cardiomyocytes are prone to multinucleation and increased ploidy following DNA synthesis, a phenomenon that contributes to the loss of regenerative capacity in mice (Patterson et al. 2017). Localization of the cytokinesis marker Aurora B can distinguish between events of complete cytokinesis and binucleation in cardiomyocytes. Symmetrical localization of Aurora B between two nuclei is indicative of cytokinesis, whereas asymmetrical localization of Aurora B can indicate binucleation (Hesse et al. 2018, Leone et al. 2015). To determine whether LRRC10 regulates later stages of cardiomyocyte cell cycle, we measured levels of the symmetric and asymmetric Aurora B localization in cardiomyocytes at 7 days post-MI. Remarkably, we detected a significant decrease in the number of symmetrically localized Aurora B cardiomyocytes in Lrrc10^{-/-} mice compared to control mice following MI (FIGS. 1D, 3C, and 3D). There was no significant difference in the number of cardiomyocytes with asymmetric Aurora B localization between WT and Lrrc10^{-/-} mice (FIG. 3E). Interestingly, at 7 days-post sham surgery, WT and Lrrc10^{-/-} mice had comparable levels of cardiomyocytes positive for pH3 (FIG. 1C), as well as symmetrical and asymmetrical Aurora B localization (FIGS. 1D and 3E), indicating that the impact of Lrrc10 on cardiomyocyte cytokinesis is an injury-specific response that is only evident during regeneration. These results suggest that LRRC10 specifically regulates cardiomyocyte cytokinesis during cardiomyocyte proliferation and heart regeneration.

To further establish the impact of Lrrc10 deletion on cardiomyocyte nucleation, we isolated cardiomyocytes from control and Lrrc10^{-/-} mice at 14-days post-injury and quantified nucleation with the DNA dye Hoechst. We measured a significant decrease in mononucleated cardiomyocytes, as well as a significant increase in binucleated and multinucleated cardiomyocytes in Lrrc10^{-/-} mice post-MI compared to control mice (FIG. 1E). No significant difference in nucleation levels were identified between sham control and Lrrc10^{-/-} mice (FIG. 3F), further confirming that Lrrc10 deletion does not impact cardiomyocyte cell cycle and nucleation in the absence of injury. Furthermore, we quantified a significant increase in cardiomyocyte size in Lrrc10^{-/-} mice compared to controls post-MI by Wheat Germ Agglutinin (WGA) staining, without significant changes in cardiomyocyte area post-sham surgery (FIG. 1F).

Collectively, our results demonstrate that Lrrc10 deletion specifically impedes cardiomyocyte cytokinesis but not karyokinesis following injury, which results in increased cardiomyocyte nucleation and blockade of the neonatal cardiac regenerative response. Thus, LRRC10 plays a conserved role in heart regeneration across permissive species in controlling the late stages of cardiomyocyte proliferation prior to completion of cell division.

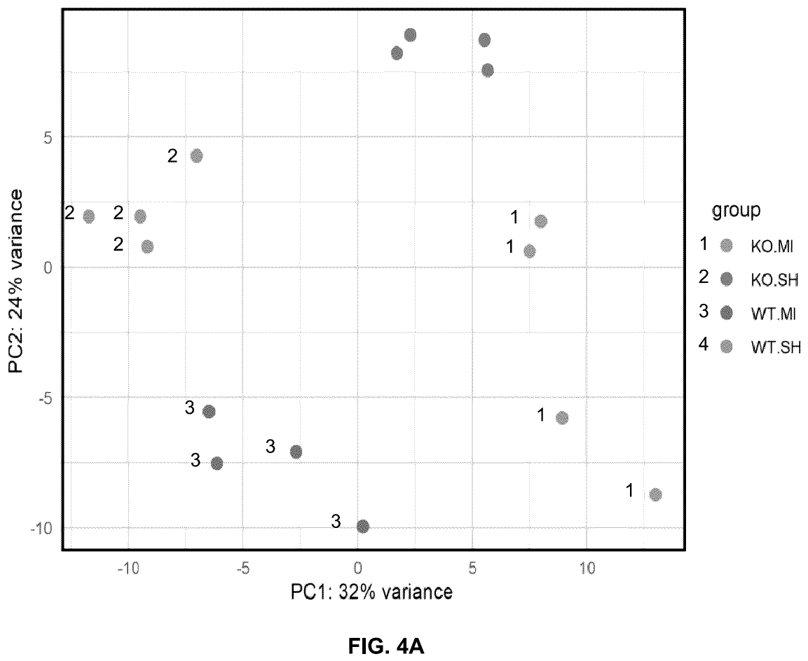

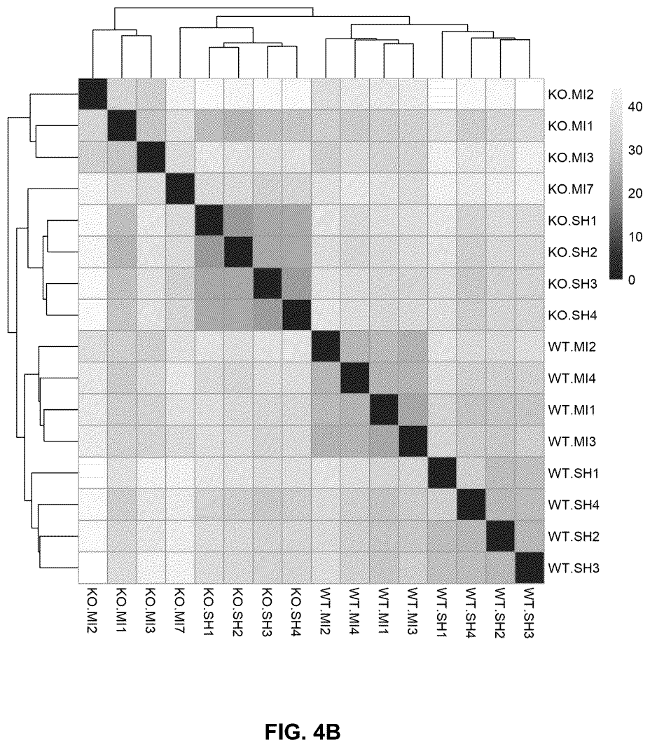

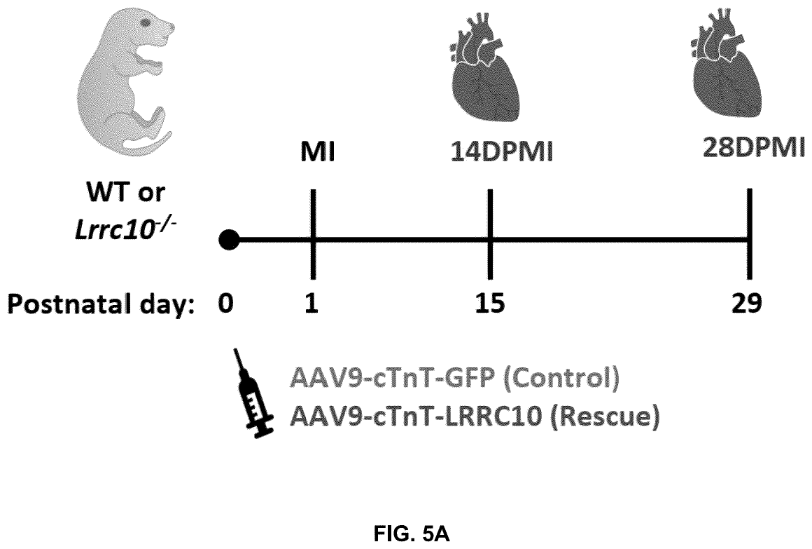

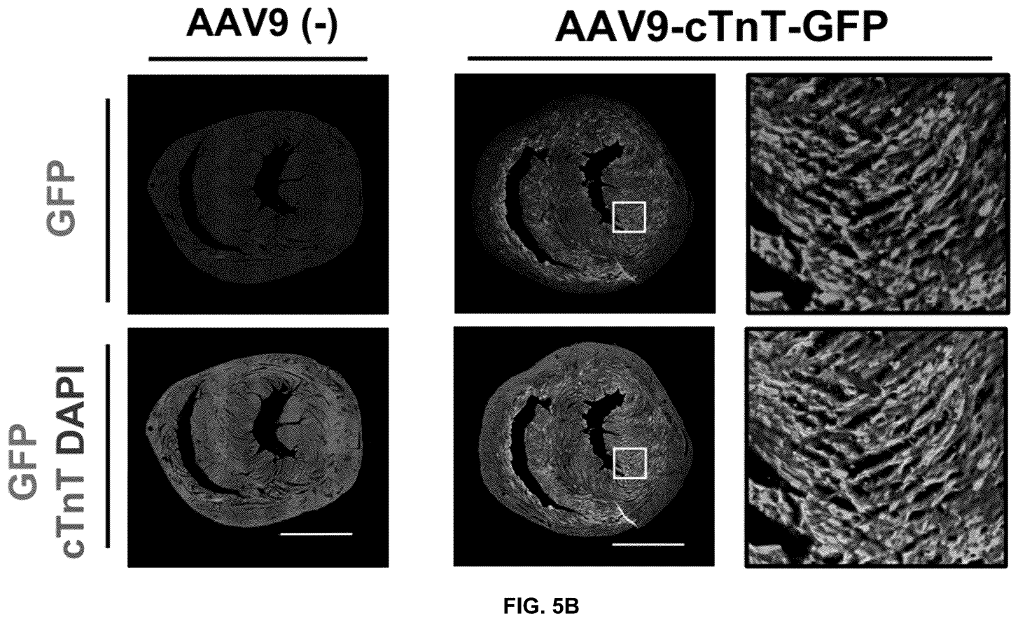

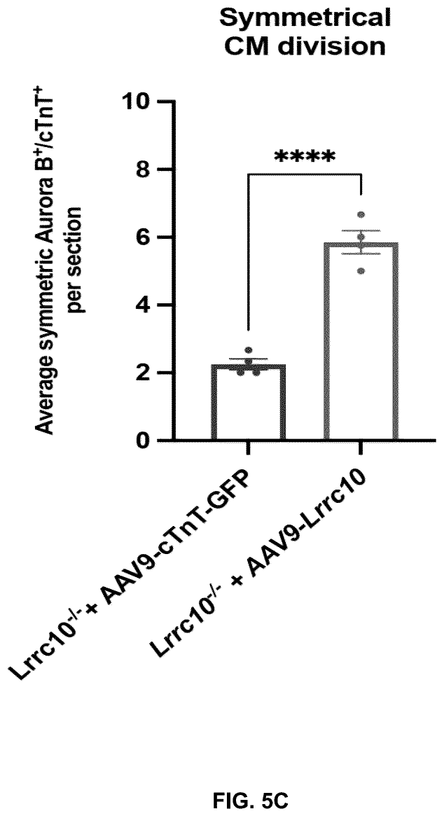

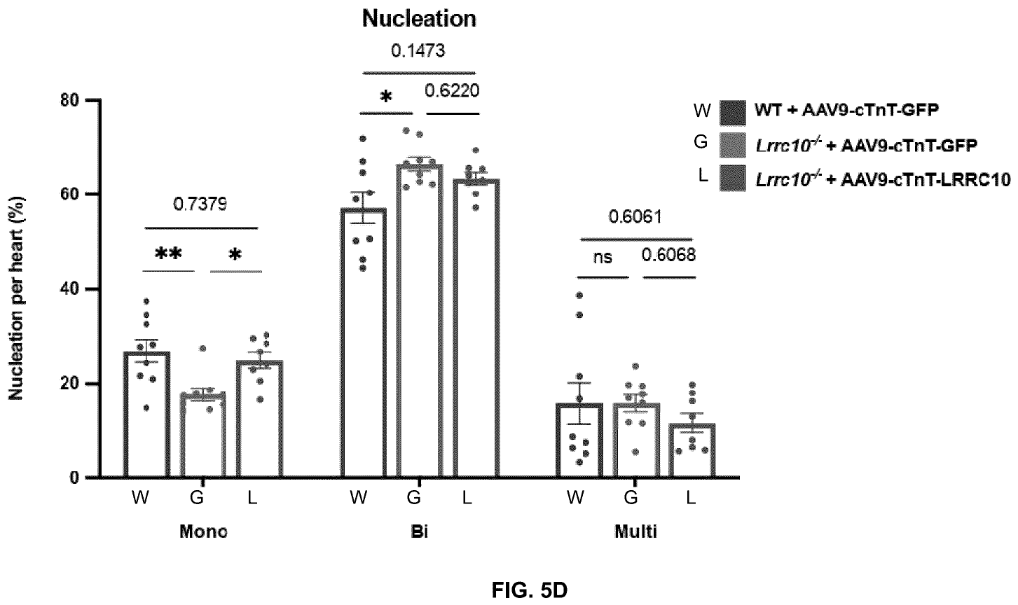

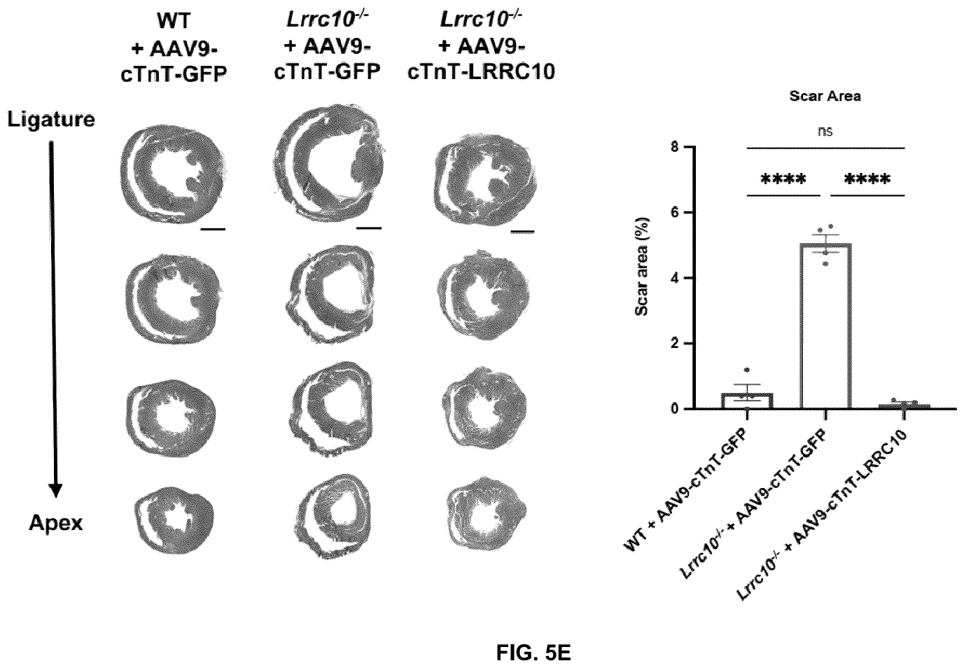

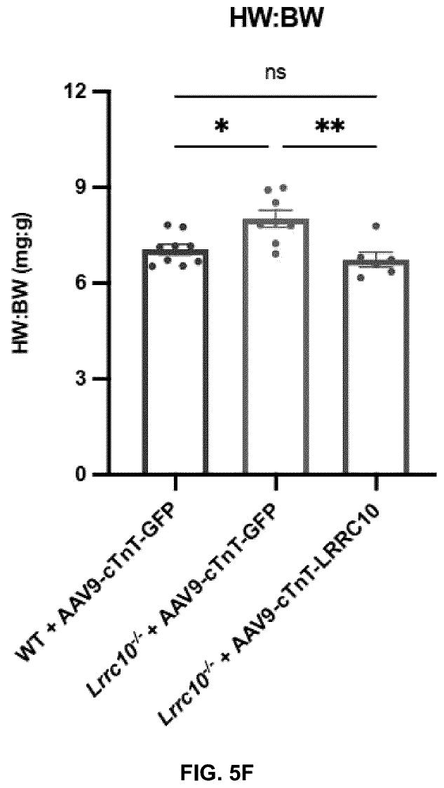

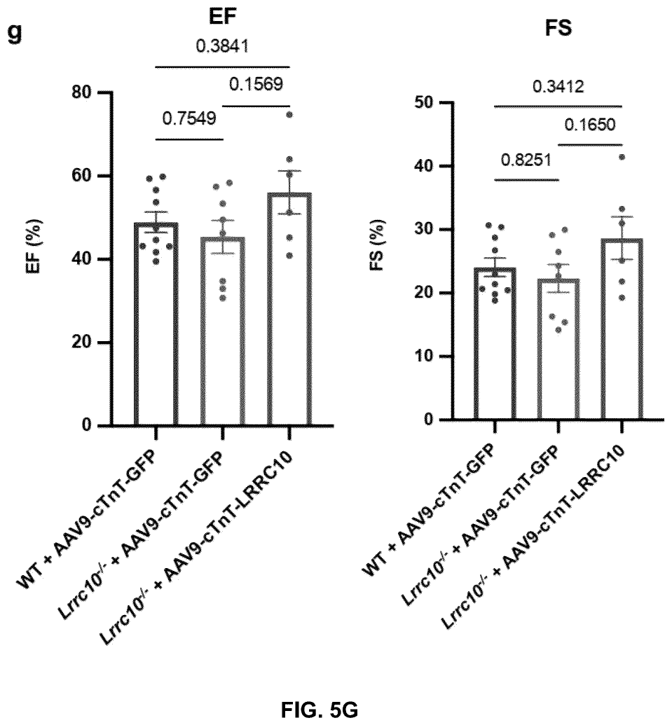

Our results demonstrate that LRRC10 regulates cardiomyocyte cytokinesis and mammalian heart regeneration post-MI. The endogenous heart regenerative response of the neonatal mouse heart is regulated by a unique transcriptional landscape (Cui et al. 2020, Quaife-Ryan et al. 2017, O'Meara et al. 2015). To identify how LRRC10 mediates cardiomyocyte cytokinesis and cardiac regeneration at the transcriptional level, we analyzed the global transcriptome in control and Lrrc10^{-/-} hearts. We performed bulk RNA sequencing on the ventricles from WT control and Lrrc10^{-/-} mice that underwent a sham or MI surgery at P1 (FIGS. 1G-1J). Hearts were collected at 7 days post-injury from equal numbers of male and female mice. Analysis of the transcriptomic landscape by Principal Component Analysis (PCA) and Pearson correlation demonstrates distinct clustering between control and Lrrc10^{-/-} sham and MI groups (FIGS. 4A and 4B), indicative of a unique transcriptional signature in Lrrc10^{-/-} mice following injury.

To further dissect the transcriptional signature related to the regenerative defects following Lrrc10 deletion, we defined the differentially expressed genes (DEGs) and performed K-means clustering and Gene Ontology (GO) analysis of the DEGs. We identified 8 total clusters with distinct transcriptomic patterns (FIG. 1G). Most interestingly, Cluster 1 shows a transcriptomic signature only upregulated in the Lrrc10^{-/-} mice post-MI (FIG. 1G, Cluster 1). The clustered genes encode for myofilament proteins, such as myosins (Myh1, Myh3, Myh8), troponins (Tnnt3, Tnnc2, Tnni2), and actin (Actn3) proteins (FIG. 1H). Furthermore, GO analysis identified cell processes related to regulation of muscle contraction and development (FIGS. 1G-1J). This increased expression of myofilament proteins maybe a compensatory effect, as recent evidence demonstrates that LRRC10 modulates Caᵥ1.2 calcium channels and cardiomyocyte contraction (Woon et al. 2018), while LRRC10 deletion results in development of dilated cardiomyopathy later in life (Brody et al. 2012).

Cluster 3 shows a distinct transcriptome in both sham and MI Lrrc10^{-/-} mice compared to controls, which includes genes encoding for major ion channels, such as a voltage-gated T-type Ca²⁺ channel (Cacnal g), sodium channels (Scna10a, Scn3b), and a potassium channel (Kcnaj5) (FIGS. 1G and 1H, Cluster 3). GO analysis related these channel-encoding genes to regulation of cardiac contraction, membrane potential, and depolarization (FIG. 1H). Changes in ion channel expression can be linked to a variety of cardiac pathologies. For example, mutations in sodium channels can lead to ventricular arrythmias in humans, and expression of T-type Ca²⁺ channel genes are associated with cardiac hypertrophy (Houser et al. 2000, Valdivia et al. 2010).

The transcriptome in Cluster 4 reveals a specific gene expression in Lrrc10^{-/-} samples post-MI compared to controls (FIG. 1G, Cluster 4), suggesting this cluster contained a distinct transcriptomic profile linked to the injury response of Lrrc10^{-/-} hearts. Further analysis identified genes associated with cell cycle such as Cdc20, Ccnb1, and the related GO pathways involved in cell cycle regulation (FIGS. 1H-1J). Interestingly, one of the downregulated genes in the cluster, Aurkb, encodes for the cytokinesis regulator Aurora B kinase, supporting our earlier evidence that Lrrc10^{-/-} inhibits the completion of cardiomyocyte division (FIG. 1D).

Lastly, we investigated the transcriptome in Cluster 7, as the overall gene expression was downregulated in Lrrc10^{-/-} sham and MI mice compared to controls. Genes and related GO terms highlight an association to cell metabolism, such as fatty acid oxidation and mitochondrial metabolism (Sdhaf1, Ndufs5, Slc25a33) (FIGS. 1G and 1H, Cluster 7). The cardiac metabolic state plays an important role in mediating endogenous heart regeneration, where altering the balance between glucose and fatty acid oxidation metabolism can promote or inhibit regeneration, respectively (Bae et al. 2021). This transcriptional signature demonstrates that Lrrc10 deletion may drive metabolic dysregulation and result in the impaired regenerative response to injury.

Our analysis highlights important roles for LRRC10 in transcriptional regulation of key cell processes related to muscle contraction, ion-channel function, cell cycle activity, and metabolism. Together, this demonstrates that Lrrc10^{-/-} mice have a unique transcriptional signature underpinning the loss of the cardiac regenerative capacity.

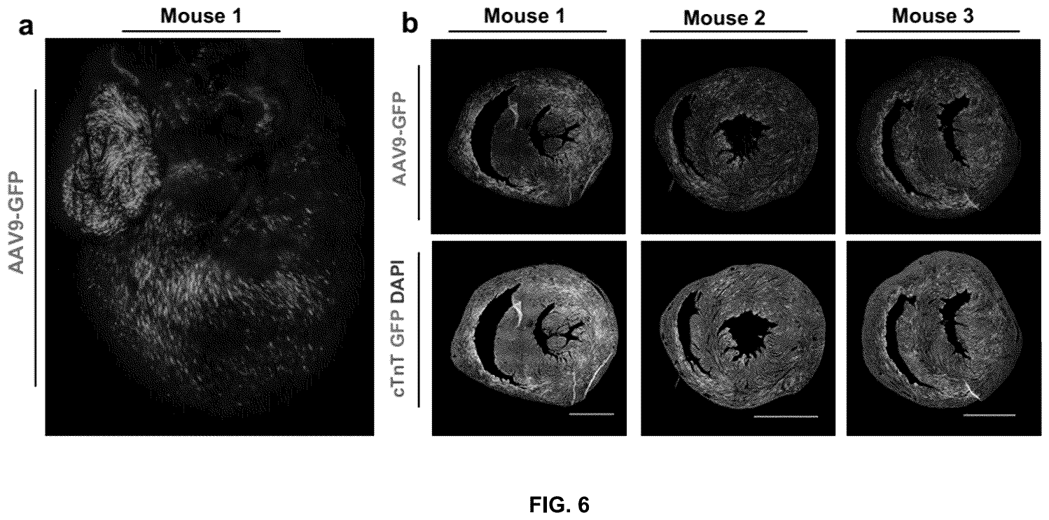

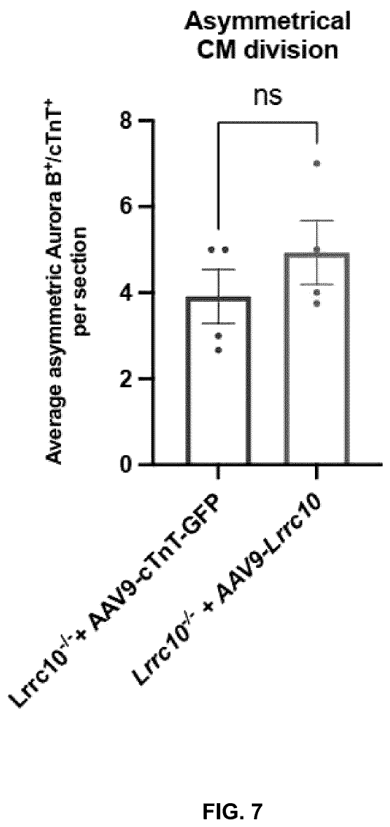

Our results demonstrate that loss of LRRC10 blocks neonatal heart regeneration. However, whether lack of LRRC10 is primarily responsible for the defect in neonatal heart regeneration rather than secondary effects on the heart remains unclear. Thus, we wanted to determine whether restoration of LRRC10 protein levels can rescue the blockade of the cardiac regenerative response in post-natal Lrrc10^{-/-} hearts. (FIG. 2A). To address this question, we injected WT and Lrrc10^{-/-} mice at P0 with a single dose of AAV9-cTnT-GFP control vector or AAV9-cTnT-LRRC10 rescue vector, for cardiac-specific overexpression of LRRC10, followed by an MI surgery at P1 (FIG. 2A). This dosing strategy was sufficient to target the heart, as evident by GFP expression throughout the heart by 7 days post-injection (FIGS. 2B and 6). We first investigated if the overexpression of LRRC10 in Lrrc10^{-/-} hearts was sufficient to rescue the defect of symmetrical Aurora B localization in cardiomyocytes. Interestingly, at 7 days post-MI we measured a significant increase in cytokinesis in Lrrc10^{-/-} mice treated with AAV9-cTnT-LRRC10 rescue vector compared to the AAV9-cTnT-GFP control vector (FIG. 2C). There was no significant difference in asymmetrical Aurora B localization between the same groups (FIG. 7), in line with our earlier results demonstrating comparable levels of asymmetrical Aurora B between WT and Lrrc10^{-/-} hearts post-MI(FIG. 1D).

We further investigated the effect of LRRC10 overexpression on cardiomyocyte cytokinesis by quantifying cardiomyocyte nucleation as a readout of cardiomyocyte division across control and rescue groups. At 14 days post-MI, LRRC10 rescue hearts restored the increase in mononucleated cardiomyocytes compared to the LRRC10 KO controls (FIG. 2D). Furthermore, the number of binucleated and multinucleated cardiomyocytes in LRRC10 rescue hearts were restored to the levels of WT control hearts, whereas LRRC10 KO controls show a significant elevation in binucleated cardiomyocytes (FIG. 2D). This demonstrates that overexpression of LRRC10 in Lrrc10^{-/-} mice restores the increase in mononucleated cardiomyocytes post-MI.

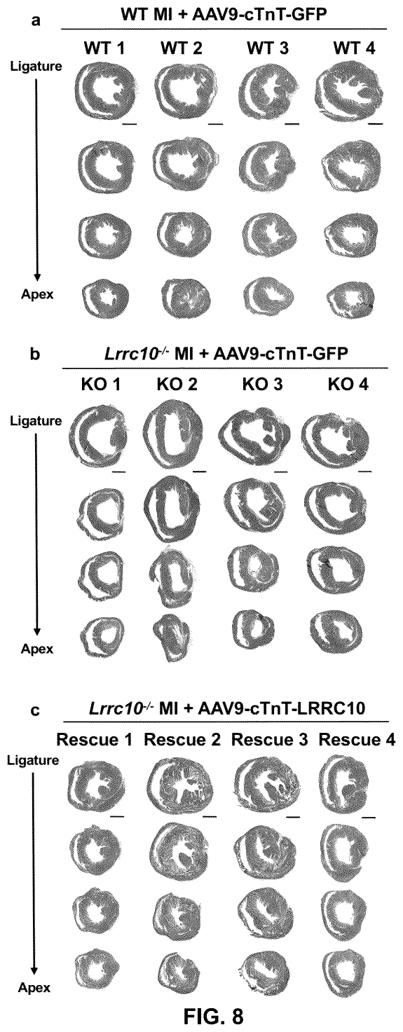

To determine whether LRRC10 overexpression restores myocardial regeneration and scar size reduction in Lrrc10^{-/-} mice, we performed trichrome staining at 28 days post-MI. LRRC10 KO control mice show incomplete regeneration, persistent scar tissue, and thinning of the left ventricle, as expected (FIGS. 2E and 8). Strikingly, the LRRC10 rescue mice show structural regeneration, with little to no scarring present and increased wall thickness in the left ventricle similar to WT controls. In addition, LRRC10 rescue mice also demonstrate a reduction of heart weight to body weight ratio, restoring heart size to similar levels as WT controls (FIG. 2F). To identify if this repair translated to improved cardiac function, we used echocardiography to measure ejection fraction (EF) and fractional shortening. Hearts of LRRC10 rescue mice showed trending improvements in EF and FS compared to LRRC10 KO control hearts (FIG. 2G). The restoration of myocardial structure and scar resolution demonstrate that LRRC10 overexpression can promote heart regeneration in Lrrc10^{-/-} hearts (FIGS. 2E-2G). Together, these results demonstrate that the inhibition of regeneration in Lrrc10^{-/-} mice is driven by reduced levels of LRRC10 and that LRRC10 overexpression postnatally is sufficient to restore myocardial regeneration following neonatal MI.

Heart failure with reduced ejection fraction following myocardial infarction remains a major health and economic burden given the inability of the adult mammalian heart to regenerate following injury. Defining the mechanisms that control endogenous heart regeneration can identify new therapeutic approaches to promote adult human heart regeneration. Our results demonstrate an evolutionarily conserved role for LRRC10 in heart regeneration, from the Astyanax mexicanus surface fish and zebrafish to the neonatal mouse. More importantly, we demonstrate that LRRC10 regulates later stages of cardiomyocyte cell cycle activity that impacts cardiomyocyte division and nucleation during neonatal mouse heart regeneration. Loss of LRRC10 results in transcriptional dysregulation of muscle, ion channel, cell cycle, and metabolic genes, which may play a role in impeding heart regeneration. Remarkably, restoration of LRRC10 levels in Lrrc10^{-/-} hearts is sufficient to rescue the endogenous regenerative response. Our results suggest a unique role for LRRC10 in regulating cardiomyocyte cytokinesis but not karyokinesis, demonstrating a stage-specific regulation of cardiomyocyte cell cycle.

To show that increased LRRC10 expression in the adult heart can induce a cardiac regenerative response following adult MI in WT mice, we can perform MI in 2-3-month-old WT mice. The day following MI, intravenous injection by tail vein can be performed with a titer of AAV9-cTnT-LRRC10, AAV9-cTnT-NLS-LRRC10 (which harbors a nuclear localization signal (NLS) to target LRRC10 in the nucleus), AAV9-cTnT-NLS-eGFP (control), or PBS (buffer control). Each experimental group can include 10 mice based on prior studies and statistical power calculations. Hearts can be harvested at 2- and 4-weeks post-MI. We can quantify the scar size by performing trichrome staining at 4 weeks post-MI. Cardiac function can be measured by serial echocardiography at multiple timepoints including 2- and 4-weeks post-MI to define the impact of LRRC10 overexpression on enhancing adult cardiac function over time post-MI.

To further show the role of LRRC10 in promoting adult cardiomyocyte proliferation, we can perform a BrdU pulse-chase experiment by adding BrdU (0.25 mg/ml) to the drinking water for 2 weeks post-MI in 3-month-old LRRC10 overexpression and control mice. Hearts can be harvested at 2 weeks post-MI, where we can co-stain BrdU with the cardiomyocyte nuclear marker PCM1 to quantify the BrdU-positive cardiomyocytes. To quantify cardiomyocyte nucleation and ploidy following LRRC10 overexpression, we can isolate adult cardiomyocytes from 3-month-old WT mice treated with the AAV9-cTnT-LRRC10 or AAV9-cTnT-NLS-LRRC10 post-MI. Cardiomyocytes can be isolated at 2- and 4-weeks post-MI as described recently (Ackers-Johnson 2016). We can quantify cardiomyocyte ploidy by performing DNA fluorescence in situ hybridization (FISH) using 2 independent autosomal probes at 14- and 28-days post-MI. This approach can establish that LRRC10 overexpression in the adult heart can promote adult cardiomyocyte proliferation.

To show that increased nuclear LRRC10 expression in the adult heart can induce a cardiac regenerative response following adult infarction in WT and postnatal LRRC10 deletion, we can perform MI in 3-month-old WT and Lrrc10^{-/-} mice, followed by injection of AAV9-cTnT-NLS-LRRC10 intravenously, which harbors a nuclear localization signal (NLS) to target LRRC10 in the nucleus. WT mice injected with AAV9-cTnT-NLS-GFP can be used as controls. We can utilize mosaic analysis with double marker (MADM) mice (Zong et al. 2005 for clonal analysis and quantification of cardiomyocyte cytokinesis and proliferation. Hearts can be harvested at 2- and 4-weeks post-MI. We can quantify the scar size by performing trichrome staining at 4 weeks post-MI. Cardiac function can be measured by serial echocardiography at multiple timepoints including 2- and 4-weeks post-MI to define the impact of LRRC10 overexpression on enhancing adult cardiac function over time post-MI.

To show that LRRC10 overexpression promotes heart regeneration in pigs, we can overexpress LRRC10 starting 3 days following MI in adult pigs. We can test delivery of either AAV9-cTnT-GFP-LRRC10 or AAV9-cTnT-GFP-NLS-LRRC10. The establishment of infarction in adult pigs can be confirmed by cardiac magnetic resonance imaging with late gadolinium enhancement (cMRI with LGE) at 3 days post-MI. Following cMRI and confirmation of a dense infarction of >8% of the left ventricle (LV) or an ejection fraction (EF)<45%, the experimental animals can be randomly assigned to two experimental groups: AAV9-cTnT-GFP-(NLS)-LRRC10 low dose (n=2) or AAV9-cTnT-GFP-(NLS)-LRRC10 high dose (n=2). The vectors can be delivered by intracoronary injection into the infarct associated vessel (left anterior descending) to target virus to the heart and region for repair/regeneration. This targeted delivery allows a lower dose per kg compared to mice where intravenous delivery is used. Based on the literature, we will test 1×10¹³ vg (low dose) and 3×10¹³ vg (high dose).

To quantify myocardial regeneration in pigs following LRRC10 overexpression, trichrome staining can be performed on histological sections from harvested hearts at 60 days post-MI to quantify fibrotic scar size in control and LRRC10 overexpression treated pigs. In addition, measurement of cardiac muscle mass, infarct size, and function of infarcted pig hearts can be assessed using cMRI with LGE at 3- and 60-days post-MI as previously described (Gabisonia et al. 2019). We can measure global left ventricular functional parameters such as ejection fraction, end-diastolic, and end-systolic volume. This can determine the ability of LRRC10 overexpression to progressively restore cardiac function over time following adult pig MI. To show that LRRC10 overexpression can stimulate cardiomyocyte proliferation, we can inject BrdU from day 3 to day 14 post-MI. Hearts can be harvested at 60 days post-MI, sectioned, and stained for BrdU and the cardiomyocyte nuclear marker PCM1 as previously described (Gabisonia et al. 2019). These results will show the role of LRRC10 overexpression in promoting pig cardiomyocyte proliferation.

This study provides an important new target to modulate cardiomyocyte cell cycle activity and heart regeneration.

### Methods

### Animals

Wild type C57BL/6J (Stock #000664) mice were obtained from Jackson Laboratories. Lrrc10^{-/-} mice were generated in C57BL/6 background and genotyped as described previously (Brody et al. 2012, Manuylov et al. 2008). All animal experimental procedures were approved by the Institutional Animal Care and Use Committee of the University of Wisconsin-Madison. All experiments were performed on age and sex matched mice, and RNA-seq analysis was performed with an equal ratio of male to female mice.

### Neonatal Myocardial Infarction

Neonatal mice underwent myocardial infarction (MI) surgery at postnatal day 1, as previously described (Mahmoud et al. 2014). Briefly, neonates were anesthetized by hypothermia on ice. A blunt dissection was performed in the fourth intercostal space. The heart was gently guided to rest on the chest cavity and the LAD was located. Using a C-1 tapered needle with a 6-0 Prolene suture (Ethicon Inc., Bridgewater, NJ), the LAD was ligated and blanching at the apex was visualized. The heart was guided back into the chest, the ribs were sutured closed, and skin was joined using adhesive glue (3M). The mice recovered on a warmed heating pad until mobile. The sham operation consisted of hypothermic anesthesia, blunt dissection in the fourth intercostal space, and closing of the chest cavity, without heart exposure or LAD ligation.

### Histology

For paraffin embedding, hearts were fixed in 4% paraformaldehyde (PFA) in PBS at 4° C. overnight. Samples were embedded in a paraffin block and sectioned below the ligation at 5 um thickness. Masson's trichrome stain was run according to the manufacturer's protocol (Newcomer Supply, Middleton, WI). Scar area quantified in ImageJ and averaged across 3 sections per heart.

For cryosections, hearts were fixed in 4% PFA in PBS at RT for 1 hr. Tissues were soaked in 30% sucrose overnight before being submerged into cryomold with OTC and frozen at −80° C. Hearts were sectioned at 8 um thickness.

### Immunostaining

Paraffin sections underwent deparaffinization and rehydration by sequential 3 min incubations in xylene and ethanol (100%, 90%, 70%) solutions. Samples were placed into IHC antigen retrieval solution (Invitrogen, Carlsbad, CA) and microwaved for 10 min. Sections were blocked in 10% blocking serum (matching secondary) and incubated in primary antibodies overnight incubation at 4° C. Primary antibodies were used against phospho-Histone3 Ser10 (Millipore, catalog #06-570) at [1:200] dilution, Aurora B (Sigma, catalog #A5102) at [1:100] dilution, and Cardiac Troponin T (cTnt Abcam, catalog #AB8295) at [1:200] dilution. Sections were washed with PBS and incubated with secondary antibodies (Invitrogen) at [1:400] dilution with DAPI for 1 hour at room temperature. Slides were mounted in antifade mounting medium and stored at 4° C. Representative images were taken on a Nikon AIRS HD confocal microscope.

Cardiomyocyte cross-sectional area was measured by Wheat Germ Agglutinin (WGA) staining. Paraffin sections were processed as described above, incubating with WGA-488 conjugated antibody (Thermo Fisher, catalog #W11261) at [1:50] dilution and cTnT. Cardiomyocyte area was quantified by measuring cross-sectional area of cardiomyocytes dual-positive for WGA+ and cTnT in Image J, measuring approximately 200 cardiomyocytes across 4-6 replicate sections.

For GFP staining in AAV9-injected mice, cryosections were placed into a humidifying chamber and incubated with 10% blocking buffer, diluted in PBS with 0.2% Triton X-100 (PBST) at RT for 1 hr. Slides were incubated with primary GFP-488 conjugated antibody (Thermo Fisher, catalog #A21331) and cTnT diluted in PBST with 5% blocking buffer at 4° C. overnight. Sections were washed, mounted, and stored at −20° C. Representative images were taken on a Nikon AIRS HD confocal microscope. For whole-mount staining, the whole heart was harvested, washed in PBS and immediately imaged for endogenous GFP expression. Representative images were acquired on a Nikon Upright FN1 confocal microscope.

### AAV9 Injection

Adenovirus vector constructs, AAV9-cTnT-EGFP-WPRE (catalog #VB5428) and AAV9-cTnT-mLRRC10-WPRE were designed and produced by Vector Biolabs (Malvern, PA). Mice were injected subcutaneously at P0 with AAV9 constructs at a viral titer of 5×10¹³ vg/kg BW (diluted in saline to a total volume of 10 ul). MI was performed at P1, as described above, and hearts were collected at 7 days-post MI for cytokinesis analysis, 14 days post-MI for nucleation analysis, and 21 days post-MI for histological analysis.

### Cardiomyocyte Isolation and Nucleation

Mice hearts were harvested at 14 days post-surgery and fixed in 4% PFA in PBS at RT for 2 hrs. Hearts were washed for three, 15 min incubations in PBS. Hearts were mined into 1 mm pieces and transferred into Eppendorf tubes containing collagenase solution with collagenase D (2.4 mg/ml, Cat #11088866001) and collagenase B (1.8 mg/ml, Cat #: #110088807007) diluted in Hank's Balanced Salt Solution (Santa Cruz, #sc-391061A) and incubated on a rocker at 37° C. overnight. Collagenase solution was replaced every two days by centrifugation at 500 g for 1 min at RT and removing supernatant. After cells were dissociated, cell pellets were collected by centrifugation at 500 g for 2 min at RT, resuspended in PBS, and passed through a 100 μm cell filter to purify cardiomyocyte populations and remove clumps. Cells were stored at 4° C. until ready for staining.

Nucleation staining and quantification was performed on isolated cardiomyocytes. Cells were mixed gently to resuspend and 200 ul of cardiomyocytes were transferred to a 1.5 ml Eppendorf tube with 300 ul of PBST (PBS with 0.2% Triton X-100). DAPI was added to solution and incubated for 10 min at RT. After, 1 ml of 10% blocking buffer was added to reduce cell clumping. Cells were collected by centrifugation at 800 g for 2 min and supernatant was discarded, leaving approximately 100 ul of solution. For nucleation quantification, around 300-500 cardiomyocytes were aliquoted onto a coverslip and sealed with nail polish. Approximately 1000 intact cardiomyocytes were counted per heart, with 4-9 replicate hearts per sample group. Nucleation distribution was presented as percent nucleation of total cardiomyocytes per sample.

### RNA Sequencing and Analysis

Sex-matched heart ventricles were collected at 7 days post-surgery (Sham or MI) and immediately homogenized in Trizol (Invitrogen) according to the manufacturer's protocol. Mouse tissue samples suspended in TRIzol were submitted to the University of Wisconsin Biotechnology Center (UWBC) Gene Expression Center (Research Resource Identifier—RRID:SCR_017757) for RNA extraction. Total RNA was purified following the recommendations of the Qiagen RNeasy Mini (Qiagen, Hilden, Germany) procedure, which included on-column DNase treatment. RNA quality and integrity (RINe>8.4) were verified on a NanoDrop One Spectrophotometer (Thermo Fisher Scientific, Waltham, MA, USA) and Agilent 4200Tapestation (Santa Clara, CA, USA), respectively.

Total RNA was used as input material and libraries were prepared by following the SMARTer Stranded Total RNA Sample Prep Kit—HI Mammalian user manual (Takara Bio USA, Mountain View, CA, USA). In brief, 900 ng total RNA were hybridized to RiboGone™ oligos for depletion of rRNA sequences by RNase H-mediated digestion followed by SPRI bead cleanup. Reduced rRNA templates were fragmented at 94° C. for 3 min prior to first-strand synthesis. Takara adaptors and indexes were added to single-stranded cDNA via 12 cycles of PCR. Quality and quantity of the finished libraries were assessed on the Agilent 4200 Tapestation (Agilent, Santa Clara, CA, USA) and Qubit Fluorometer (Invitrogen, Carlsbad, CA, USA), respectively. Paired end 150 bp sequencing was performed by Illumina Sequencing by UWBC DNA Sequencing Facility (RRID:SCR_017759) on an Illumina NovaSeq6000, with libraries multiplexed for an approximate 50 million reads per library. Sequencing was done using standard 300 cycle TruSeq v1.5 SBS kits and SCS 2.8 software. Images were analyzed using the standard Illumina Pipeline, version 1.8.

Bioinformatic analysis of transcriptomic data adhere to recommended ENCODE guidelines and best practices for RNA-Seq (Encode Consortium, 2016). Alignment of adapter-trimmed (Jiang et al. 2014) (Skewer v0.1.123) 2×150 (paired-end; PE) bp strand-specific Illumina reads to the Mus musculus Mus musculus GRCm39 mouse genome (assembly accession NCBI: GCA_000001635.9) was achieved with the Spliced Transcripts Alignment to a Reference (STAR v2.5.3a) software (Dobin et al. 2013), a splice-junction aware aligner. Expression estimation was performed with RSEM (Li et al. 2011) (RNASeq by Expectation Maximization, v1.3.0), generating overall RSEM gene counts. Counts were normalized by TPM. To test for differential gene expression among individual group contrasts, expected read counts obtained from RSEM were used as input into DESeq2 (Love et al. 2014) (Version 1.36.0). Statistical significance of Differentially Expressed genes (DEGs) was defined by a log 2fold change of ±(0.5), with statistical significance of the negative-binomial regression test adjusted with a Benjamini-Hochberg FDR correction at the 10% level (Reiner et al. 2003) and independent filtering requiring genes to have a minimum read count (10 reads) in each group. Heatmap was generated using pheatmap [V.1.0.12] from K-means clustering of all DEGs in the 4-way comparison, with cell value is TPM row-normalized. DOT plots for GO analysis were generated with enrichGO in Clusterprofiler using p-value <0.05. These raw and processed data sets have been deposited in NCBI's Gene Expression Omnibus and are accessible through GEO accession number GSE221539. Groups for RNA-seq were sex matched and independently analyzed, with no sex-specific differences identified.

### Statistical Analysis

Graphs were generated using Prism 9 (GraphPad Software). Statistical analysis between two groups was run using a student's unpaired t-test. Multiple groups were compared using ordinary one-way ANOVA with Tukey post hoc test to determine significant comparisons. Statistical significance described as a p<0.05. P-values shown as *(P≤0.05). **(P≤0.01), ***(P≤0.001), ****(P≤0.0001). n.s. indicates not significant. Error bars presented as S.E.M.

### Data Availability

RNA-seq data from WT and Lrrc10′ mice are available at the NCBI's Gene Expression Omnibus (GSE221539).

### REFERENCES

Ackers-Johnson M, Li P Y, Holmes A P, O'Brien S M, Pavlovic D and Foo R S. A Simplified, Langendorff-Free Method for Concomitant Isolation of Viable Cardiac Myocytes and Nonmyocytes From the Adult Mouse Heart. Circ Res. 2016; 119:909-20.
Adameyko I I, Mudry R E, Houston-Cummings N R, Veselov A P, Gregorio C C, Tevosian S G. Expression and regulation of mouse SERDINI, a highly conserved cardiac-specific leucine-rich repeat protein. Dev Dyn. 2005 June; 233(2):540-52.
Andries O., Mc Cafferty S., De Smedt S. C., Weiss R., Sanders N. N., Kitada T. N(1)-methylpseudouridine-incorporated mRNA outperforms pseudouridine-incorporated mRNA by providing enhanced protein expression and reduced immunogenicity in mammalian cell lines and mice. J. Control. Release. 2015; 217:337-344.
Bae, J. et al. Malonate Promotes Adult Cardiomyocyte Proliferation and Heart Regeneration. Circulation 143, 1973-1986, (2021).
Bassat E., Mutlak Y. E., Genzelinakh A., Shadrin I. Y., Baruch Umansky K., Yifa O., Kain D., Rajchman D., Leach J., Riabov Bassat D. The extracellular matrix protein agrin promotes heart regeneration in mice. Nature. 2017; 547:179-184.
Becker, R. O., Chapin, S. & Sherry, R. Regeneration of the ventricular myocardium in amphibians. Nature 248, 145-147, (1974).
Bersell K, Arab S, Haring B, Kuhn B. Neuregulin1/ErbB4 signaling induces cardiomyocyte proliferation and repair of heart injury. Cell. 2009 Jul. 23; 138(2):257-70.
Boecker W, Bernecker O Y, Wu J C, Zhu X, Sawa T, Grazette L, Rosenzweig A, del Monte F, Schmidt U, Hajjar R J. Cardiac-specific gene expression facilitated by an enhanced myosin light chain promoter. Mol Imaging. 2004 April; 3(2):69-75.
Brody, M. J. et al. Ablation of the cardiac-specific gene leucine-rich repeat containing 10 (Lrrc10) results in dilated cardiomyopathy. PLoS One 7, e51621, (2012).
Bui A. L., Horwich T. B., Fonarow G. C. Epidemiology and risk profile of heart failure. Nat. Rev. Cardiol. 2011; 8:30-41.
Cano-Martinez, A. et al. Functional and structural regeneration in the axolotl heart (Ambystoma mexicanum) after partial ventricular amputation. Arch Cardiol Mex 80, 79-86(2010).
Carlsson L., Clarke J. C., Yen C., Gregoire F., Albery T., Billger M., Egnell A. C., Gan L. M., Jennbacken K., Johansson E. Biocompatible, purified VEGF-A mRNA improves cardiac function after intracardiac injection 1 week post-myocardial infarction in Swine. Mol. Ther. Methods Clin. Dev. 2018; 9:330-346.
Chen S. J., Johnston J., Sandhu A., Bish L. T., Hovhannisyan R., Jno-Charles O., Sweeney H. L., Wilson J. M. Enhancing the utility of adeno-associated virus gene transfer through inducible tissue-specific expression. Hum. Gene Ther. Methods. 2013; 24:270278.
Chu D., Sullivan C. C., Weitzman M. D., Du L., Wolf P. L., Jamieson S. W., Thistlethwaite P. A. Direct comparison of efficiency and stability of gene transfer into the mammalian heart using adeno-associated virus versus adenovirus vectors. J. Thorac. Cardiovasc. Surg. 2003; 126:671-679.
Crozat K., Beutler B. TLR7: a new sensor of viral infection. Proc. Natl. Acad. Sci. USA. 2004; 101:6835-6836.
Cui, M. et al. Dynamic Transcriptional Responses to Injury of Regenerative and Non-regenerative Cardiomyocytes Revealed by Single-Nucleus RNA Sequencing. Dev Cell 53, 102-116 e108, (2020).
Dargie H. Heart failure post-myocardial infarction: a review of the issues. Heart. 2005; 91(Suppl 2):ii3-ii6. discussion ii31, ii43-ii48. del Monte F., Hajjar R. J., Harding S. E. Overwhelming evidence of the beneficial effects of SERCA gene transfer in heart failure. Circ. Res. 2001; 88:E66-E67.
Dobin, A. et al. STAR: ultrafast universal RNA-seq aligner. Bioinformatics 29, 15-21, (2013).
Engel F. B., Hsieh P. C., Lee R. T., Keating M. T. FGF1/p38 MAP kinase inhibitor therapy induces cardiomyocyte mitosis, reduces scarring, and rescues function after myocardial infarction. Proc. Natl. Acad. Sci. USA. 2006; 103:15546-15551.
Engel F. B., Schebesta M., Duong M. T., Lu G., Ren S., Madwed J. B., Jiang H., Wang Y., Keating M. T. p38 MAP kinase inhibition enables proliferation of adult mammalian cardiomyocytes. Genes Dev. 2005; 19:1175-1187.
Eulalio A., Mano M., Dal Ferro M., Zentilin L., Sinagra G., Zacchigna S., Giacca M. Functional screening identifies miRNAs inducing cardiac regeneration. Nature. 2012; 492:376-381.
Fiedorowicz K, Rozwadowska N, Zimna A, Malcher A, Tutak K, Szczerbal I, Nowicka-Bauer K, Nowaczyk M, Kolanowski T J, Labȩdź W, Kubaszewski L, Kurpisz M. Tissue-specific promoter-based reporter system for monitoring cell differentiation from iPSCs to cardiomyocytes. Sci Rep. 2020 Feb. 5; 10(1):1895.
Formiga F R, Tamayo E, Simón-Yarza T, Pelacho B, Prósper F, Blanco-Prieto M J. Angiogenic therapy for cardiac repair based on protein delivery systems. Heart Fail Rev. 2012 May; 17(3):449-73.
French B. A., Mazur W., Geske R. S., Bolli R. Direct in vivo gene transfer into porcine myocardium using replication-deficient adenoviral vectors. Circulation. 1994; 90:2414-2424.
Gabisonia K, Prosdocimo G, Aquaro G D, Carlucci L, Zentilin L, Secco I, Ali H, Braga L, Gorgodze N, Bernini F, Burchielli S, Collesi C, Zandona L, Sinagra G, Piacenti M, Zacchigna S, Bussani R, Recchia F A and Giacca M. MicroRNA therapy stimulates uncontrolled cardiac repair after myocardial infarction in pigs. Nature. 2019; 569:418-422.
Gao J., Liu J., Gao Y., Wang C., Zhao Y., Chen B., Xiao Z., Miao Q., Dai J. A myocardial patch made of collagen membranes loaded with collagen-binding human vascular endothelial growth factor accelerates healing of the injured rabbit heart. Tissue Eng. Part A. 2011; 17:2739-2747.
Go A. S., Mozaffarian D., Roger V. L., Benjamin E. J., Berry J. D., Blaha M. J., Dai S., Ford E. S., Fox C. S., Franco S., American Heart Association Statistics Committee and Stroke Statistics Subcommittee Heart disease and stroke statistics-2014 update: a report from the American Heart Association. Circulation. 2014; 129:e28-e292.
Goswami R, Subramanian G, Silayeva L, Newkirk I, Doctor D, Chawla K, Chattopadhyay S, Chandra D, Chilukuri N, Betapudi V. Gene Therapy Leaves a Vicious Cycle. Front Oncol. 2019 Apr. 24; 9:297.
Greenberg B., Butler J., Felker G. M., Ponikowski P., Voors A. A., Desai A. S., Barnard D., Bouchard A., Jaski B., Lyon A. R. Calcium upregulation by percutaneous administration of gene therapy in patients with cardiac disease (CUPID 2): a randomised, multinational, double-blind, placebo-controlled, phase 2b trial. Lancet. 2016; 387:1178-1186.
Hadas Y., Katz M. G., Bridges C. R., Zangi L. Modified mRNA as a therapeutic tool to induce cardiac regeneration in ischemic heart disease. Wiley Interdiscip. Rev. Syst. Biol. Med. 2017; 9:e1367.
Hajjar R. J., Zsebo K., Deckelbaum L., Thompson C., Rudy J., Yaroshinsky A., Ly H., Kawase Y., Wagner K., Borow K. Design of a phase ½ trial of intracoronary administration of AAV1/SERCA2a in patients with heart failure. J. Card. Fail. 2008; 14:355-367.
Hedman M, Hartikainen J, Syvänne M, Stjernvall J, Hedman A, Kivelä A, Vanninen E, Mussalo H, Kauppila E, Simula S, Närvänen O, Rantala A, Peuhkurinen K, Nieminen M S, Laakso M, Ylä-Herttuala S. Safety and feasibility of catheter-based local intracoronary vascular endothelial growth factor gene transfer in the prevention of postangioplasty and in-stent restenosis and in the treatment of chronic myocardial ischemia: phase II results of the Kuopio Angiogenesis Trial (KAT). Circulation. 2003 Jun. 3; 107(21):2677-83.
Hendel R. C., Henry T. D., Rocha-Singh K., Isner J. M., Kereiakes D. J., Giordano F. J., Simons M., Bonow R. O. Effect of intracoronary recombinant human vascular endothelial growth factor on myocardial perfusion: evidence for a dose-dependent effect. Circulation. 2000; 101:118-121.
Henry T. D., Annex B. H., McKendall G. R., Azrin M. A., Lopez J. J., Giordano F. J., Shah P. K., Willerson J. T., Benza R. L., Berman D. S., VIVA Investigators The VIVA trial: vascular endothelial growth factor in ischemia for vascular angiogenesis. Circulation. 2003; 107:1359-1365.
Hermans-Beijnsberger S., van Bilsen M., Schroen B. Long non-coding RNAs in the failing heart and vasculature. Noncoding RNA Res. 2018; 3:118-130.
Hesse M, Doengi M, Becker A, Kimura K, Voeltz N, Stein V, Fleischmann B K. Midbody Positioning and Distance Between Daughter Nuclei Enable Unequivocal Identification of Cardiomyocyte Cell Division in Mice. Circ Res. 2018 Oct. 12; 123(9):1039-1052.
Hinkel R., Trenkwalder T., Kupatt C. Gene therapy for ischemic heart disease. Expert Opin. Biol. Ther. 2011; 11:723-737.
Houser, S. R., Piacentino, V., 3rd & Weisser, J. Abnormalities of calcium cycling in the hypertrophied and failing heart. J Mol Cell Cardiol 32, 1595-1607, (2000).
Huang C L, Leblond A L, Turner E C, Kumar A H, Martin K, Whelan D, O'Sullivan D M, Caplice N M. Synthetic chemically modified mrna-based delivery of cytoprotective factor promotes early cardiomyocyte survival post-acute myocardial infarction. Mol Pharm. 2015 Mar. 2; 12(3):991-6.
Ieda M., Fu J. D., Delgado-Olguin P., Vedantham V., Hayashi Y., Bruneau B. G., Srivastava D. Direct reprogramming of fibroblasts into functional cardiomyocytes by defined factors. Cell. 2010; 142:375-386.
Jiang, H., Lei, R., Ding, S.-W. & Zhu, S. Skewer: a fast and accurate adapter trimmer for next-generation sequencing paired-end reads. BMC Bioinformatics 15, 182, (2014).
Karikó K, Muramatsu H, Welsh F A, Ludwig J, Kato H, Akira S, Weissman D. Incorporation of pseudouridine into mRNA yields superior nonimmunogenic vector with increased translational capacity and biological stability. Mol Ther. 2008 November; 16(11):1833-40.
Kho C., Lee A., Jeong D., Oh J. G., Chaanine A. H., Kizana E., Park W. J., Hajjar R. J. SUMO1-dependent modulation of SERCA2a in heart failure. Nature. 2011; 477:601-605.
Kim K H, Antkiewicz D S, Yan L, Eliceiri K W, Heideman W, Peterson R E, Lee Y. Lrrc10 is required for early heart development and function in zebrafish. Dev Biol. 2007 Aug. 15; 308(2):494-506.
Kim K H, Kim T G, Micales B K, Lyons G E, Lee Y. Dynamic expression patterns of leucine-rich repeat containing protein 10 in the heart. Dev Dyn. 2007 August; 236(8):2225-34.
Kobe, B. & Deisenhofer, J. The leucine-rich repeat: a versatile binding motif. Trends Biochem Sci 19, 415-421, (1994).
Kondrat J., Sultana N., Zangi L. Synthesis of modified mRNA for myocardial delivery. Methods Mol. Biol. 2017; 1521:127138.
Kormann M. S., Hasenpusch G., Aneja M. K., Nica G., Flemmer A. W., Herber-Jonat S., Huppmann M., Mays L. E., Illenyi M., Schams A. Expression of therapeutic proteins after delivery of chemically modified mRNA in mice. Nat. Biotechnol. 2011; 29:154-157.
Korpela H, Järveläinen N, Siimes S, Lampela J, Airaksinen J, Valli K, Turunen M, Pajula J, Nurro J, Yla-Herttuala S. Gene therapy for ischaemic heart disease and heart failure. J Intern Med. 2021 September; 290(3):567-582.
Kühn B, del Monte F, Hajjar R J, Chang Y S, Lebeche D, Arab S, Keating M T. Periostin induces proliferation of differentiated cardiomyocytes and promotes cardiac repair. Nat Med. 2007 August; 13(8):962-9.
Lee C J, Fan X, Guo X, Medin J A. Promoter-specific lentivectors for long-term, cardiac-directed therapy of Fabry disease. J Cardiol. 2011 January; 57(1):115-22.
Leone M, Magadum A, Engel F B. Cardiomyocyte proliferation in cardiac development and regeneration: a guide to methodologies and interpretations. Am J Physiol Heart Circ Physiol. 2015 October; 309(8):H1237-50.
Li B, Dewey C N. RSEM: accurate transcript quantification from RNA-Seq data with or without a reference genome. BMC Bioinformatics. 2011 Aug. 4; 12:323.
Li J., Yousefi K., Ding W., Singh J., Shehadeh L. A. Osteopontin RNA aptamer can prevent and reverse pressure overload-induced heart failure. Cardiovasc. Res. 2017; 113:633-643.
Lin Y D, Luo C Y, Hu Y N, Yeh M L, Hsueh Y C, Chang M Y, Tsai D C, Wang J N, Tang M J, Wei E I, Springer M L, Hsieh P C. Instructive nanofiber scaffolds with VEGF create a microenvironment for arteriogenesis and cardiac repair. Sci Transl Med. 2012 Aug. 8; 4(146):146ra109.
Lin Z, Pu W T. Strategies for cardiac regeneration and repair. Sci Transl Med. 2014 Jun. 4; 6(239):239rv1.
Lin Z., von Gise A., Zhou P., Gu F., Ma Q., Jiang J., Yau A. L., Buck J. N., Gouin K. A., van Gorp P. R. Cardiac-specific YAP activation improves cardiac function and survival in an experimental murine MI model. Circ. Res. 2014; 115:354-363.
Losordo D. W., Vale P. R., Symes J. F., Dunnington C. H., Esakof D. D., Maysky M., Ashare A. B., Lathi K., Isner J. M. Gene therapy for myocardial angiogenesis: initial clinical results with direct myocardial injection of phVEGF165 as sole therapy for myocardial ischemia. Circulation. 1998; 98:2800-2804.
Love, M. I., Huber, W. & Anders, S. Moderated estimation of fold change and dispersion for RNA-seq data with DESeq2. Genome Biology 15, 550, (2014).
Lui K. O., Zangi L., Silva E. A., Bu L., Sahara M., Li R. A., Mooney D. J., Chien K. R. Driving vascular endothelial cell fate of human multipotent Is11+heart progenitors with VEGF modified mRNA. Cell Res. 2013; 23:1172-1186.
Magadum A, Kaur K, Zangi L. mRNA-Based Protein Replacement Therapy for the Heart. Mol Ther. 2019 Apr. 10; 27(4):785-793.
Magadum A., Singh N., Kurian A. A., Sharkar M. T. K., Chepurko E., Zangi L. Ablation of a single N-glycosylation site in human FSTL 1 induces cardiomyocyte proliferation and cardiac regeneration. Mol. Ther. Nucleic Acids. 2018; 13:133-143.
Mahmoud, A. I., Porrello, E. R., Kimura, W., Olson, E. N. & Sadek, H. A. Surgical models for cardiac regeneration in neonatal mice. Nature Protocols 9, 305-311, (2014).
Manuylov, N. L., Manuylova, E., Avdoshina, V. & Tevosian, S. Serdin1/Lrrc10 is dispensable for mouse development. Genesis 46, 441-446, (2008).
Mingozzi F., High K. A. Immune responses to AAV vectors: overcoming barriers to successful gene therapy. Blood. 2013; 122:23-36.
Mockey M., GonÃ§ alves C., Dupuy F. P., Lemoine F. M., Pichon C., Midoux P. mRNA transfection of dendritic cells: synergistic effect of ARCA mRNA capping with Poly(A) chains in cis and in trans for a high protein expression level. Biochem. Biophys. Res. Commun. 2006; 340:1062-1068.
Murphy, S. P., Ibrahim, N. E. & Januzzi, J. L., Jr. Heart Failure With Reduced Ejection Fraction: A Review. JAMA 324, 488-504, (2020).
Muruve D. A. The innate immune response to adenovirus vectors. Hum. Gene Ther. 2004; 15:1157-1166.
Nagy E., Jermendy A. L., Merkely B., Maurovich-Horvat P. Clinical importance of epicardial adipose tissue. Arch. Med. Sci. 2017; 13:864-874.
O'Meara, C. C. et al. Transcriptional reversion of cardiac myocyte fate during mammalian cardiac regeneration. Circ Res 116, 804-815, (2015).
Papadakis E. D., Nicklin S. A., Baker A. H., White S. J. Promoters and control elements: designing expression cassettes for gene therapy. Curr. Gene Ther. 2004; 4:89-113.
Patterson, M. et al. Frequency of mononuclear diploid cardiomyocytes underlies natural variation in heart regeneration. Nat Genet 49, 1346-1353, (2017).
Phillips M I, Tang Y, Schmidt-Ott K, Qian K, Kagiyama S. Vigilant vector: heart-specific promoter in an adeno-associated virus vector for cardioprotection. Hypertension. 2002 February; 39(2 Pt 2):651-5.
Porrello, E. R. et al. Regulation of neonatal and adult mammalian heart regeneration by the miR-15 family. Proc Natl Acad Sci USA 110, 187-192, (2013).
Porrello E. R., Mahmoud A. I., Simpson E., Hill J. A., Richardson J. A., Olson E. N., Sadek H. A. Transient regenerative potential of the neonatal mouse heart. Science. 2011; 331:1078-1080.
Poss, K. D., Wilson, L. G. & Keating, M. T. Heart regeneration in zebrafish. Science 298, 2188-2190, (2002).
Quaife-Ryan, G. A. et al. Multicellular Transcriptional Analysis of Mammalian Heart Regeneration. Circulation 136, 1123-1139, (2017).
Rane S., He M., Sayed D., Vashistha H., Malhotra A., Sadoshima J., Vatner D. E., Vatner S. F., Abdellatif M. Downregulation of miR-199a derepresses hypoxia-inducible factor-1alpha and Sirtuin 1 and recapitulates hypoxia preconditioning in cardiac myocytes. Circ. Res. 2009; 104:879-886.
Reiner, A., Yekutieli, D. & Benjamini, Y. Identifying differentially expressed genes using false discovery rate controlling procedures. Bioinformatics 19, 368-375, (2003).
Ren G., Dewald O., Frangogiannis N. G. Inflammatory mechanisms in myocardial infarction. Curr. Drug Targets Inflamm. Allergy. 2003; 2:242-256.
Sadek, H. & Olson, E. N. Toward the Goal of Human Heart Regeneration. Cell Stem Cell 26, 7-16, (2020).
Salamon R J, McKeon M C, Bae J, Zhang X, Paltzer W G, Wanless K N, Schuett A R, Nuttall D J, Nemr S A, Sridharan R, Lee Y, Kamp T J, Mahmoud A I. LRRC10 regulates mammalian cardiomyocyte cell cycle during heart regeneration. NPJ Regen Med. 2023 Jul. 28; 8(1):39.
Sato K., Wu T., Laham R. J., Johnson R. B., Douglas P., Li J., Sellke F. W., Bunting S., Simons M., Post M. J. Efficacy of intracoronary or intravenous VEGF165 in a pig model of chronic myocardial ischemia. J. Am. Coll. Cardiol. 2001; 37:616-623.
Savarese G., Lund L. H. Global public health burden of heart failure. Card. Fail. Rev. 2017; 3:7-11.
Simón-Yarza T, Formiga F R, Tamayo E, Pelacho B, Prosper F, Blanco-Prieto M J. Vascular endothelial growth factor-delivery systems for cardiac repair: an overview. Theranostics. 2012; 2(6):541-52.
Shapiro S D, Ranjan A K, Kawase Y, Cheng R K, Kara R J, Bhattacharya R, Guzman-Martinez G, Sanz J, Garcia M J, Chaudhry H W. Cyclin A2 induces cardiac regeneration after myocardial infarction through cytokinesis of adult cardiomyocytes. Sci Transl Med. 2014 Feb. 19; 6(224):224ra27.
Singh R. D., Hillestad M. L., Livia C., Li M., Alekseev A. E., Witt T. A., Stalboerger P. G., Yamada S., Terzic A., Behfar A. M3RNA drives targeted gene delivery in acute myocardial infarction. Tissue Eng. Part A. 2018 Published online Sep. 21, 2018.
Song K., Nam Y. J., Luo X., Qi X., Tan W., Huang G. N., Acharya A., Smith C. L., Tallquist M. D., Neilson E. G. Heart repair by reprogramming non-myocytes with cardiac transcription factors. Nature. 2012; 485:599-604.
Stewart D. J., Hilton J. D., Arnold J. M., Gregoire J., Rivard A., Archer S. L., Charbonneau F., Cohen E., Curtis M., Buller C. E. Angiogenic gene therapy in patients with nonrevascularizable ischemic heart disease: a phase 2 randomized, controlled trial of AdVEGF(121) (AdVEGF121) versus maximum medical treatment. Gene Ther. 2006; 13:1503-1511.
Stewart D. J., Kutryk M. J., Fitchett D., Freeman M., Camack N., Su Y., Della Siega A., Bilodeau L., Burton J. R., Proulx G., Radhakrishnan S., NORTHERN Trial Investigators VEGF gene therapy fails to improve perfusion of ischemic myocardium in patients with advanced coronary disease: results of the NORTHERN trial. Mol. Ther. 2009; 17:1109-1115.
Stockdale, W. T. et al. Heart Regeneration in the Mexican Cavefish. Cell Rep 25, 1997-2007 e1997, (2018).
Sultana N., Magadum A., Hadas Y., Kondrat J., Singh N., Youssef E., Calderon D., Chepurko E., Dubois N., Hajjar R. J., Zangi L. Optimizing cardiac delivery of modified mRNA. Mol. Ther. 2017; 25:1306-1315.
Svitkin Y V, Cheng Y M, Chakraborty T, Presnyak V, John M, Sonenberg N. N1-methyl-pseudouridine in mRNA enhances translation through eIF2α-dependent and independent mechanisms by increasing ribosome density. Nucleic Acids Res. 2017 Jun. 2; 45(10):6023-6036.
Turnbull I. C., Eltoukhy A. A., Anderson D. G., Costa K. D. Lipidoid mRNA nanoparticles for myocardial delivery in rodents. Methods Mol. Biol. 2017; 1521:153-166.
Turnbull I. C., Eltoukhy A. A., Fish K. M., Nonnenmacher M., Ishikawa K., Chen J., Hajjar R. J., Anderson D. G., Costa K. D. Myocardial delivery of lipidoid nanoparticle carrying modRNA induces rapid and transient expression. Mol. Ther. 2016; 24:66-75.
Valdivia, C. R. et al. Loss-of-function mutation of the SCN3B-encoded sodium channel beta3 subunit associated with a case of idiopathic ventricular fibrillation. Cardiovasc Res 86, 392-400, (2010).
Vassalli G, Biieler H, Dudler J, von Segesser L K, Kappenberger L. Adeno-associated virus (AAV) vectors achieve prolonged transgene expression in mouse myocardium and arteries in vivo: a comparative study with adenovirus vectors. Int J Cardiol. 2003 August; 90(2-3):229-38.
Wei K, Serpooshan V, Hurtado C, Diez-Cuñado M, Zhao M, Maruyama S, Zhu W, Fajardo G, Noseda M, Nakamura K, Tian X, Liu Q, Wang A, Matsuura Y, Bushway P, Cai W, Savchenko A, Mahmoudi M, Schneider M D, van den Hoff M J, Butte M J, Yang P C, Walsh K, Zhou B, Bernstein D, Mercola M, Ruiz-Lozano P. Epicardial FSTL1 reconstitution regenerates the adult mammalian heart. Nature. 2015 Sep. 24; 525(7570):479-85.
Wolff J. A., Malone R. W., Williams P., Chong W., Acsadi G., Jani A., Felgner P. L. Direct gene transfer into mouse muscle in vivo. Science. 1990; 247:1465-1468.
Woon, M. T. et al. Pediatric Dilated Cardiomyopathy-Associated LRRC10 (Leucine-Rich Repeat-Containing 10) Variant Reveals LRRC10 as an Auxiliary Subunit of Cardiac L-Type Ca(2+) Channels. J Am Heart Assoc 7, (2018).
Wu J., Zeng F., Huang X. P., Chung J. C., Konecny F., Weisel R. D., Li R. K. Infarct stabilization and cardiac repair with a VEGF-conjugated, injectable hydrogel. Biomaterials. 2011; 32:579-586.
Ye, L. et al. Early Regenerative Capacity in the Porcine Heart. Circulation 138, 2798-2808, (2018).
Yu S, Yang H, Li T, Pan H, Ren S, Luo G, Jiang J, Yu L, Chen B, Zhang Y, Wang S, Tian R, Zhang T, Zhang S, Chen Y, Yuan Q, Ge S, Zhang J, Xia N. Efficient intracellular delivery of proteins by a multifunctional chimaeric peptide in vitro and in vivo. Nat Commun. 2021 Aug. 26; 12(1):5131.
Zacchigna S., Zentilin L., Giacca M. Adeno-associated virus vectors as therapeutic and investigational tools in the cardiovascular system. Circ. Res. 2014; 114:1827-1846.
Zangi, L., K. Kaundal, R., & Kaur, K. (2021). Gene Therapy for Heart Disease: Modified mRNA Perspectives. Cardiomyopathy—Disease of the Heart Muscle.
Zangi L, Lui K O, von Gise A, Ma Q, Ebina W, Ptaszek L M, Später D, Xu H, Tabebordbar M, Gorbatov R, Sena B, Nahrendorf M, Briscoe D M, Li R A, Wagers A J, Rossi D J, Pu W T, Chien K R. Modified mRNA directs the fate of heart progenitor cells and induces vascular regeneration after myocardial infarction. Nat Biotechnol. 2013 October; 31(10):898-907.
Zangi L, Oliveira M S, Ye L Y, Ma Q, Sultana N, Hadas Y, Chepurko E, Später D, Zhou B, Chew W L, Ebina W, Abrial M, Wang Q D, Pu W T, Chien K R. Insulin-Like Growth Factor 1 Receptor-Dependent Pathway Drives Epicardial Adipose Tissue Formation After Myocardial Injury. Circulation. 2017 Jan. 3; 135(1):59-72.
Zhou J., Rossi J. J. Cell-type-specific, aptamer-functionalized agents for targeted disease therapy. Mol. Ther. Nucleic Acids. 2014; 3:e169.
Zhu, W. et al. Regenerative Potential of Neonatal Porcine Hearts. Circulation 138, 2809-2816, (2018).
Zong H, Espinosa J S, Su H H, Muzumdar M D and Luo L. Mosaic analysis with double markers in mice. Cell. 2005; 121:479-92.

1. A method of inducing mammalian cardiac cell proliferation, the method comprising ectopically introducing an LRRC10 protein in a mammalian cardiac cell to thereby induce proliferation of the cardiac cell.

2. The method of claim 1, wherein the cardiac cell comprises a cardiomyocyte.

3. The method of claim 1, wherein the LRRC10 protein is at least 95% identical to SEQ ID NO:1.

4. (canceled)

5. The method of claim 1, wherein the ectopically introducing comprises expressing the LRRC10 protein from a recombinant nucleic acid encoding the LRRC10 protein within the cell.

6. The method of claim 1, wherein the ectopically introducing comprises delivering an exogenous nucleic acid encoding the LRRC10 protein in the cardiac cell.

7-8. (canceled)

9. The method of claim 1, wherein the cardiac cell is comprised within a heart.

10. The method of claim 9, wherein the heart comprises a cardiac injury.

11. The method of claim 10, wherein the cardiac injury comprises a myocardial lesion, wherein the myocardial lesion is selected from the group consisting of a myocardial infarct, decreased myocardial thickness, fibrosis, dilated cardiac chamber, increased left ventricular end-diastolic diameter, increased left ventricular end-systolic diameter, and any combination thereof.

12. (canceled)

13. The method of claim 10, wherein the cardiac injury comprises a functional defect, wherein the functional defect is selected from the group consisting of reduced ejection fraction, reduced fractional shortening, reduced end-systolic elastance, and any combination thereof.

14. (canceled)

15. The method of claim 10, wherein the LRRC10 protein is introduced in an amount and for a time effective to elicit an amelioration of the cardiac injury.

16. The method of claim 9, wherein the LRRC10 protein is introduced in an amount and for a time effective to elicit an improvement in cardiac structure, wherein the improvement in cardiac structure is selected from the group consisting of a decrease in myocardial lesion size, an increase in myocardial thickness, decreased fibrosis, a decrease in left ventricular end-diastolic diameter, a decrease in left ventricular end-systolic diameter, an increase in coronary artery formation, an increase in capillary density, an increase in revascularization, and any combination thereof.

17. (canceled)

18. The method of claim 9, wherein the LRRC10 protein is introduced in an amount and for a time effective to elicit an improvement in cardiac function, wherein the improvement in cardiac function is selected from the group consisting of an increase in an increase in ejection fraction, an increase in fractional shortening, an increase in end-systolic elastance, and any combination thereof.

19. (canceled)

20. The method of claim 1, wherein the cardiac cell is comprised within a subject.

21. The method of claim 20, wherein the subject is suffering or has suffered from a cardiac event or a chronic heart condition.

22. The method of claim 21, wherein the subject is suffering or has suffered from a cardiac event, wherein the cardiac event is selected from the group consisting of cardiac ischemia, cardiac ischemia-reperfusion, myocardial infarction, myocarditis, blunt trauma, and any combination thereof.

23. (canceled)

24. The method of claim 21, wherein the subject is suffering or has suffered from a chronic heart condition.

25. The method of claim 20, wherein the subject is suffering from or has suffered from heart failure.

26. The method of claim 25, wherein the heart failure comprises heart failure with reduced ejection fraction.

27. The method of claim 20, wherein the subject has a myocardial infarct.

28. The method of claim 20, comprising administering a nucleic-acid delivery reagent comprising an ectopic nucleic acid to the subject.


SEQUENCE LISTING (2 sequences)

SEQ ID NO: 1 - protein, 277 aa, Homo sapiens
MGNTIRALVAFIPADRCQNYVVRDLREMPLDKMVDLSGSQLRRFPLHVCSFRELVKLYLSDNHLNSLPPELGQLQNLQILALDFNNFKALPQVVCTLKQLCILYLGNNKLCDLPSELSLLQNLRTLWIEANCLTQLPDVVCELSLLKTLHAGSNALRLLPGQLRRLQELRTIWLSGNRLTDFPTVLLHMPFLEVIDVDWNSIRYFPSLAHLSSLKLVIYDHNPCRNAPKVAKGVRRVGRWAEETPEPDPRKARRYALVREESQELQAPVPLLPPTNS

SEQ ID NO: 2 - DNA, 834 bp, Homo sp.
  source 1..834: mol_type = other DNA
atggggaacaccatcagggccctcgtggccttcatccctgctgaccgttgccagaactatgtggtcagggacctccgtgagatgccgctggacaagatggtggatctgagtgggagccagttacgccgcttccccctgcacgtgtgctccttcagggagctggtcaagctctacctgagcgacaaccacctcaatagcctgcctccggagctggggcagctacagaacctgcagattctggccttggatttcaacaacttcaaggctctgccccaggtggtgtgcaccttgaaacagctctgcatcctctacctgggcaacaacaaactctgcgacctccccagtgagctgagcctgctccagaacctcaggaccctgtggatcgaggccaactgcctcacccagctgccggatgtggtctgtgagctgagtctccttaagactctgcatgccggctccaacgccctgcgtttgctgccaggccagctccggcgcctccaggagctgaggaccatctggctctcgggcaaccggctaactgactttcccactgtgctgcttcacatgcccttcctggaggtgattgatgtggactggaacagcatccgttacttccccagcctggcgcacctgtcaagtctgaagctggtcatctatgaccacaatccttgcaggaacgcacccaaggtggccaaaggtgtgcgccgtgtggggagatgggcagaggagacgccagagcccgaccctagaaaagccaggcgctatgcgttggtcagagaggaaagccaggagctacaggcaccagtccctctacttcctcctaccaactcctga